(19) United States
(12) Patent Application Publication
(10) Pub. No.: US 2003/0059461 A1
(43) Pub. Date: Mar. 27, 2003

(54) Molecular delivery vehicle for delivery of selected compounds to targets

(75) Inventors: Marina V. Backer, W. Simsbury, CT (US); Joseph M. Backer, W. Simsbury, CT (US)

Correspondence Address: Docket Coordinator, WIGGIN & DANA, One Century Tower, 265 Church Street, New Haven, CT, 06508-1832, US

(73) Assignee: SibTech, Inc.

(21) Appl. No.: 09/872,712

(22) Filed: Jun. 1, 2001

Related U.S. Application Data
(60) Provisional application No. 60/209,660, filed on Jun. 6, 2000.

(51) Int. Cl. A61K 39/395; A61K 31/715; A61K 38/16; A61K 9/127; A61K 9/14

(52) U.S. Cl. 424/450; 424/178.1; 514/12; 424/486; 424/93.6; 514/54

(57) ABSTRACT

The present invention is directed to a molecular delivery vehicle for delivery of compounds to a target, comprising: (a) a carrier for carrying the compounds; (b) an adapter covalently linked to the carrier; and (c) a targeting protein comprising a recognition portion and a targeting portion, the recognition portion capable of binding to the adapter, the targeting portion capable of binding to the target. The present invention is also directed to pharmaceutical compositions including the above molecular delivery vehicle, and methods of delivering therapeutic, diagnostic, or research compounds to selected targets using the molecular delivery vehicle.

This Application claims the benefit of Provisional Application Serial No. 60/209,660 filed Jun. 6, 2000.

### STATEMENT OF GOVERNMENT SUPPORT

This invention was made in part with government support under grant number 1 R43 HL61143-01 from the National Institutes of Health. The government has certain rights in this invention.

### BACKGROUND OF THE INVENTION

1. Field of the Invention

The present invention relates generally to molecular delivery vehicles for delivery of therapeutic, diagnostic, or research compounds to a various targets on natural and/or artificial surfaces, and methods of delivering selected compounds to a target. More particularly, the present invention relates to molecular delivery vehicles for delivery of therapeutic, diagnostic, or research compounds to a target, comprising: (a) a carrier for carrying the compounds; (b) an adapter covalently linked to the carrier; and (c) a targeting protein comprising a recognition portion and a targeting portion, the recognition portion capable of binding to the adapter, the targeting portion capable of binding to the target. The present invention also relates to pharmaceutical compositions containing the molecular delivery vehicles, as well as nucleic acid and protein sequences, vectors, and transformed cells containing portions of the molecular delivery vehicles.

2. Description of the Related Art

Selective delivery of therapeutic, diagnostic, and research compounds to targeted cells improves their efficacy and minimizes potentially adverse side effects. Selective delivery of proteins to targets immobilized on artificial surfaces may be employed in medical devices and biosensors. Monoclonal antibodies, metabolites, peptide hormones, cytokines, growth factors, viral, and bacteriophage particles are employed as targeting entities. Two main approaches are used to load therapeutic, diagnostic, and research compounds onto targeting entities. In the first approach, molecules intended for delivery are chemically conjugated to targeting molecules. Accumulated experience indicates that no more than 5-10 molecules can be conjugated to a single targeting molecule (reviewed recently by Dubowchik & Walker, 1999, Pharmacol. Ther. 83, 67-123 and references therein). In a second approach, a high capacity carrier for molecules intended for delivery is either chemically conjugated to targeting molecule, or is bound non-covalently to a specific group introduced into targeting molecule (e.g. biotin). Several carriers were introduced in practice, such as dextrans, synthetic polymers, and various liposomes (reviewed recently by Dubowchik & Walker, 1999, Pharmac. & Therap. 83, 67-123 and references therein). The latter approach was particularly explored in the last few years for delivery of nucleic acids to targeted cells, using various targeting molecules and various nucleic acid carriers (see, for example, U.S. Pat. No. 6,056,973 to Allen et al.; No. 5,972,901 to Ferkol et al.; No. 5,874,297 to Wu et al.; No. 5,837,533 to Boutin; No. 5,661,025 to Szoka et al.; No. 5,766,899 to Kuo et al.; and No. 5,792,645 to Beug et al.).

Both approaches, described above, rely on chemical modifications of targeting molecules and therefore should be custom-developed and optimized on a case-by-case basis. The goals of optimization include (1) increasing the number of molecules that can be loaded on a targeting carrier without inactivation of a carrier; (2) achieving the homogeneity of preparation; and (3) achieving selective and efficient release of a “payload”. It should be noted, that Beug et al. (U.S. Pat. No. 5,792,645) mentioned a possibility of expressing targeting molecule as a fusion protein containing poly-L-lysine fragment for binding of nucleic acid. However, feasibility of constructing, expressing, purifying and using such molecules has never been tested.

Recently, various viral and bacteriophage particles were employed to deliver nucleic acids into the cells (reviewed recently by Robbins & Ghivizzani, Pharmacol. Ther., 80:35-47 (1998) and references therein). Several group reported fusion of cell-specific targeting domains to viral or bacteriophage proteins in ways that lead to the expression of fusion targeting proteins on the viral or bacteriophage surface. (see, for example Wickham, T. J. et al., (1996) Nature Biotechnol. 14:1570-1573; Wickham, T. J. et al., (1996) J. Virol. 70:6831-6838; Kovesdi, I. et al., (1997) Curr. Opin. Biotechnol. 8:583-589; Einfeld, D. A. et al., (1999) J. Virol. 73:9130-9136; Poul, M. A., and Marks, J. D. (1999) J. Mol. Biol. 288:203-211; Chadwick, M. P. et al., (1999) J. Mol. Biol. 285:485-494; U.S. Pat. Nos. 5,998,192 and 5,858,743 to Russell et al.). In known disclosures either fusion targeting proteins expressed on the surface of viral and bacteriophage particles, or bispecific antibodies for recognition of standardized epitope engineered in the fusion surface protein were created on a case-by-case basis, making a development of viral or bacteriophage particles, or bispecific antibody for every new target a scientific project with an uncertain outcome.

Various devices also require proteins associated with artificial surface and presently rely on chemical conjugation of proteins to artificial surfaces. Biosensors rely on proteins (e.g. antibodies, enzymes) conjugated to appropriate surfaces that are capable of interactions with added molecules. Medical devices (e.g. pacemakers, vascular grafts, heart valves, catheters, etc.) rely on proteins conjugated to appropriate surfaces in order to minimize or to maximize interactions with components of bodily tissues and fluids. High throughput screening of compounds for interaction with specific proteins require immobilization of said proteins on surfaces of testing devices. Several methods for chemical conjugation of proteins to the artificial surfaces of biosensors and medical devices have been developed (see, for example U.S. Pat. Nos. 5,492,840; 5,853,744; and 6,033,719). These methods were developed on the case-by-case basis in order to minimize damage to the protein and to increase the homogeneity of the surface.

Thus, in the area of target-mediated delivery of therapeutic, diagnostic, and research compounds to targets on natural and artificial surfaces, and in the area of attachment proteins to artificial surface there is a need in a method that: (1) readily converts a given protein into targeting or attachable protein; (2) does not rely on destructive chemical modifications of proteins; and (3) can utilize pre-made components carrying therapeutic, diagnostic, and research compounds. The present invention is believed to be an answer to these objectives.

### SUMMARY OF THE INVENTION

In one aspect, the present invention is directed to a molecular delivery vehicle for delivery of therapeutic, diagnostic, or research compounds to a target, comprising: (a) a carrier for carrying the compounds; (b) an adapter covalently linked to the carrier; and (c) a targeting protein comprising a recognition portion and a targeting portion, the recognition portion capable of binding to the adapter, the targeting portion capable of binding to the target.

In another aspect, the present invention is directed to a pharmaceutical composition, comprising: (1) a pharmaceutically acceptable carrier; and (2) a pharmaceutically effective amount of a molecular delivery vehicle for delivery of therapeutic, diagnostic, or research compounds to a target, comprising: (a) a carrier for carrying the compounds; (b) an adapter covalently linked to the carrier; and (c) a targeting protein comprising a recognition portion and a targeting portion, the recognition portion capable of binding to the adapter, the targeting portion capable of binding to the target. Pharmaceutically acceptable carriers include water, gelatin, lactose, starch, magnesium stearate, talc, plant oils, gums, alcohol, petroleum jellies such as Vaseline, buffered saline, and combinations thereof. Additional pharmaceutically acceptable carriers are know to those of skill in the art.

In another aspect, the present invention is directed to an article of manufacture comprising packaging material and a pharmaceutical agent contained within the packaging material, wherein the pharmaceutical agent is therapeutically effective for treating pathophysiological conditions that depend on cells that can be detected or affected via target-mediated delivery of therapeutic or diagnostic compounds and wherein the packaging material comprises a label which indicates that the pharmaceutical agent can be used for treating pathophysiological conditions that depend on cells that can be detected or affected via target-mediated delivery of therapeutic or diagnostic compounds, and wherein the pharmaceutical agent comprises a pharmaceutically effective amount of a molecular delivery vehicle for delivery of therapeutic, diagnostic, or research compounds to a target, comprising: (a) a carrier for carrying the compounds; (b) an adapter covalently linked to the carrier; and (c) a targeting protein comprising a recognition portion and a targeting portion, the recognition portion capable of binding to the adapter, the targeting portion capable of binding to the target; in a pharmaceutically acceptable carrier.

In another aspect, the present invention is directed to a method for delivering therapeutic, diagnostic, or research compounds to a target, comprising the step of: administering a pharmaceutical composition comprising: (1) a pharmaceutically acceptable carrier; and (2) a pharmaceutically effective amount of a molecular delivery vehicle for delivery of therapeutic, diagnostic, or research compounds to a target, comprising: (a) a carrier for carrying the compounds; (b) an adapter covalently linked to the carrier; and (c) a targeting protein comprising a recognition portion and a targeting portion, the recognition portion capable of binding to the adapter, the targeting portion capable of binding to the target.

In yet another aspect, the present invention is directed to a composition to cover artificial surfaces with a desired fusion targeting protein, comprising depositing on said surfaces adapter entities serving as targets that interact with a recognition peptide in the fusion targeting protein.

In another aspect, the present invention is directed to an isolated nucleic acid sequence, comprising (i) a first nucleic acid sequence segment encoding 1 to 15 amino acid residue N-terminal peptide fragment (S-peptide) of bovine or human ribonuclease A, and (ii) a second nucleic acid sequence segment encoding any full-length or mutated isoform of human vascular endothelial growth factor (VEGF), wherein the isolated nucleic acid sequence codes for a fusion protein which specifically binds adapter protein recognized by the polypeptide encoded by the first nucleic acid, and specifically binds to receptors for vascular endothelial growth factor recognized by the polypeptide encoded by the second nucleic acid sequence.

In another aspect, the present invention is directed to an expression vector, comprising control elements operably linked to the above nucleic acid sequence, and a transformed bacterial cell containing the above expression vector.

The invention also provides for targeting proteins comprising the S-peptide fragment of bovine or human ribonuclease A fused via a peptide spacer to the N-terminus of isoforms of vascular endothelial growth factor such that the resulting isolated targeting proteins possess the capability to bind to adapter protein and to bind and to activate vascular endothelial growth factor KDR/flk-1 cell surface receptor.

In another aspect, the present invention is directed to isolated nucleic acid sequences encoding wild-type or mutant fragments of bovine and human ribonuclease A (S-protein), wherein the isolated nucleic acid sequences code for adapter proteins which specifically bind S-peptide fragments of ribonuclease either alone or in recombinant targeting proteins.

In another aspect, the present invention is directed to an expression vector, comprising control elements operably linked to the above nucleic acid sequence, and a transformed bacterial cell containing the above expression vector.

The invention also provides for adapter proteins comprising the S-protein fragment of bovine or human ribonuclease A such that the resulting isolated adapter proteins possess the capability to bind to S-peptide alone or in recombinant targeting proteins.

In another aspect, the present invention is directed to an isolated nucleic acid sequences, comprising (i) a first nucleic acid sequence encoding an S-protein fragment of bovine or human ribonuclease A, and (ii) a second nucleic acid sequence encoding any full-length or mutated isoform of human vascular endothelial growth factor (VEGF), wherein the isolated nucleic acid sequence codes for a fusion protein which specifically binds S-peptide alone or in fusion proteins recognized by the polypeptide encoded by the first nucleic acid, and specifically binds to receptors for vascular endothelial growth factor recognized by the polypeptide encoded by the second nucleic acid sequence.

In another aspect, the present invention is directed to an expression vector, comprising control elements operably linked to the above nucleic acid sequence, and a transformed bacterial cell containing the above expression vector.

Invention also provides for targeting proteins comprising the S-protein fragment of bovine or human ribonuclease A fused via a peptide spacer to the N-terminus of isoforms of vascular endothelial growth factor such that the resulting isolated targeting proteins possess the capability to bind to S-peptide alone or in fusion proteins and to bind and to activate vascular endothelial growth factor KDR/flk-1 cell surface receptor.

The invention also covers compositions for use in delivery of liposome-encapsulated therapeutic and diagnostic agents to cells expressing KDR/flk-1 receptors, such compositions comprising a fusion targeting protein as defined above associated with S-protein fragment of ribonuclease A conjugated to polyethylene glycolated phospholipid, and associated with agent-loaded liposome with a pharmaceutically acceptable diluent or carrier.

The invention also covers compositions for use in delivery of DNA into cells expressing KDR/flk-1 receptors, such compositions comprising a fusion targeting protein as defined above associated with S-protein fragment of bovine pancreatic ribonuclease A conjugated with polyethylenimine and loaded with DNA in an effective amount in combination with a pharmaceutically acceptable diluent or carrier.

These and other aspects will be described in more detail in the following detailed description of the invention.

### DESCRIPTION OF THE DRAWINGS

The invention will be more fully understood from the following detailed description taken in conjunction with the accompanying figures in which:

FIGS. 1A and 1B show a schematic representation of the construction of the vehicle for target-mediated delivery of therapeutic, diagnostic, or research entities to targeted cells (FIG. 11A) or artificial surfaces (FIG. 1B);

FIG. 2 is a schematic representation of VEGF fusion protein containing human or bovine S-tag (FVEGF121, hus-VEGF121), bovine S-protein fragment from aa 16 to aa 124 (16-BoS), VEGF fusion protein containing S-protein (16-BoS-VEGF), and several recombinant mutant bovine and human S-proteins (BoS and HuS, respectively) containing an additional cysteine that is not involved in disulphide bonds and therefore is suitable for chemical crosslinking to an adapter;

FIG. 3 illustrates the ability of VEGF fusion proteins containing 158 amino acid N-terminal extensions that include the S-peptide fragment of ribonuclease A to bind to KDR/flk-1 receptors. In these experiments binding of reporter ¹²⁵I-VEGF165 proteins to soluble KDR-Fc receptors was inhibited in a dose dependent manner by correct size VEGF165 proteins and by txFVEGF121, txFVEGF165, and txFVEGF189 fusion proteins (Panel A). Scatchard analysis of binding of ¹²⁵I-txFVEGF121 to 293/KDR cells (grown in 6-well plates) and that of control ¹²⁵I-VEGF165 (grown in 24-well plates) revealed that these proteins have similar number of binding sites per cell, but txFVEGF121 has approximately ten-fold lower affinity to KDR/flk-1 receptors than VEGF165 (Panels B and C);

FIG. 4 illustrates the ability of VEGF fusion proteins containing 158 amino acid N-terminal extensions that include the S-peptide fragment of ribonuclease A to induce tyrosine autophosphorylation of KDR/flk-1 receptors in 293/KDR cells and PAE/KDR cells as detected by Western blot analysis of the cell lysates with antiphosphotyrosine antibody. (Arrows indicate tyrosine phosphorylated KDR/flk-1). Panel A illustrates that 293/KDR and PAE/KDR cells express different numbers of KDR/flk-1 receptors/cell as detected by Western blot analysis of the cell lysates with rabbit polyclonal antisera developed against the cytoplasmic domain of KDR/flk-1 receptor. Panel B illustrates that tyrosine autophosphorylation of KDR/flk-1 receptors in 293/KDR cells induced in a dose-dependent manner by indicated VEGF fusion proteins and by correct size recombinant VEGF165. Panel C illustrates tyrosine autophosphorylation of KDR/flk-1 receptors in PAE/KDR cells induced by 2.5 nM of correct size recombinant VEGF165 (1), as well as by 125 nM of txFVEGF121 (2), txFVEGF165 (3), and txFVEGF189 (4).

FIG. 5 illustrates the ability of VEGF fusion proteins containing 158 amino acid N-terminal extensions that include the S-peptide fragment of ribonuclease to induce contraction of 293/KDR cells. Cells were incubated for 3 h with buffer (panel A), or with 50 nM txFVEGF121 fusion protein (panel B);

FIG. 6 illustrates the ability of complexes of S-proteins with txFVEGF121 fusion proteins containing 158 amino acid N-terminal extensions that include the S-peptide fragment of ribonuclease to induce tyrosine autophosphorylation of KDR/flk-1 receptors as detected by Western blot analysis of the cell lysates using antiphosphotyrosine antibody. Cells were treated with buffer (Lane 1); 3.5 nM VEGF165 (Lane 2); 3.5 nM VEGF165+7 nM S-protein (Lane 3); 7 nM txFVEGF121 (Lane 4); 7 nM complexes of txFVEGF121 fusion protein with S-protein fragment of ribonuclease (Lane 5); and 7 nM S-protein alone (Lane 6);

FIGS. 7A, 7B, and 7C illustrate that S-protein/PEI conjugate (FIG. 7A), S-protein/lipid conjugate (FIG. 7B) and doxorubicin-loaded liposome carrying S-protein (FIG. 7C) retain the ability to bind to S-peptide in FVEGF121 fusion protein as measured by appearance of ribonuclease activity in reaction mixture. Reaction mixtures contained various amounts of FVEGF121 and S-protein (SP), S-protein/lipid conjugate (DSPE-SP), doxorubicin-loaded liposome carrying S-protein (Lip(Dox)-SP), or S-protein/PEI conjugate (PEI-SP). Ribonuclease activity was measured with S-tag Rapid Assay Kit (Novagen, USA) according to the manufacturer's instructions.

FIG. 8 illustrates functional activities of Lip(Dox)-SP-VEGF and DNA/PEI-SP-FVEGF121 complexes. Panel A illustrates that FVEGF121 associated with doxorubicin-loaded liposome carrying S-protein retains the ability to induce KDR/flk-1 tyrosine phosphorylation in 293/KDR cells; Panel B illustrates that FVEGF121 associated with DNA-loaded SP-PEI conjugate retains the ability to induce KDR/flk-1 tyrosine phosphorylation in 293/KDR cells. Tyrosine phosphorylation of KDR/flk-1 receptors in 293/KDR cells incubated with buffer control (lane 1); 50 ng/ml VEGF165 (lane 2); DNA/SP-PEI-FVEGF121 complexes at indicated concentrations of FVEGF121 (lanes 3-5). Panel C illustrates that DNA/PEI-SP-FVEGF121 complexes compete with ¹²⁵I-VEGF165 as efficiently as FVEGF121 for binding to cellular KDR/flk-1 receptors in 293/KDR cells. In these experiments 24 h after plating 293/KDR cells were shifted to serum-free DMEM buffered with 25 mM HEPES pH 7.5. ¹²⁵I-VEGF165 (40,000 cpm/well) was mixed with the indicated amounts of FVEGF121 or DNA/PEI-SP-FVEGF121, and added to cells washed with phosphate-buffered saline in triplicates. After a 90-minute incubation at room temperature cells were washed twice with ice-cold phosphate-buffered saline containing 1% bovine serum albumin and once with ice-cold phosphate-buffered saline containing 1% bovine serum albumin and 0.4 M NaCl. ¹²⁵I-radioactivity of the cell lysates was counted in a gamma-counter;

FIG. 9 illustrates differences between effects of FVEGF121-mediated delivery of doxorubicin-loaded liposomes on 293/KDR cells overexpressing KDR/flk-1 receptors and control 293 cells. 293/KDR and 293 parental cells were plated on 24-well plates, 5×10³ cells/well. Lip(Dox)-SP-FVEGF121 complexes with 1:1 (Panel A) or varying (Panel B) Lip(Dox)-SP:VEGF molar ratio were added to cells in fresh culture medium 20 hrs later. Control cells were treated with equal amounts of Lip(Dox)-SP without VEGF. After 16-18 hours the medium was replaced with fresh culture medium. Cells were counted in a Coulter Counter after a 4-day incubation;

FIG. 10 illustrates differences between VEGF-mediated DNA delivery to 293/KDR cells overexpressing KDR/flk-1 receptors and control 293 cells. Panel A illustrates DNA delivery initiated in serum-free medium. In these experiments DNA/PEI-SP and DNA/PEI-SP-FVEGF121 complexes were prepared at an N/P ratio of 3.75 and added to 293/KDR or to 293 cells to final concentrations of 30 nM FVEGF121, 64 nM PEI-SP conjugate, and 2 μg/well DNA in serum-free DMEM. After a 40 minute incubation at 4° C., fetal bovine serum was added to wells to a final concentration of 10%. After a 24-hour incubation at 37° C. cells were lysed and luciferase expression in cell lysates was measured using a commercially available Dual-Luciferase Reporter Assay System (Promega, USA) according to the manufacturer's instructions. Panel B illustrates DNA delivery initiated in medium containing 10% fetal bovine serum. In these experiments DNA/PEI-SP-FVEGF121 complexes containing varying amounts of FVEGF121 (28.5; 57, and 114 nM final concentrations), and fixed amounts of conjugate (64 nM final concentration) and DNA (2 μg/well) were added to cells maintained in DMEM supplemented with 10% fetal bovine serum at 37° C. After a 24-hour incubation at 37° C. cells were lysed and luciferase expression in cell lysates was measured using a commercially available Dual-Luciferase Reporter Assay System (Promega, USA) according to the manufacturer's instructions. The transfectability of 293 and 293/KDR cells was determined with DNA/PEI complexes (the N/P ratio of 8) in every experiment and found to be similar within experimental errors of 15%; and

FIG. 11 illustrates FVEGF-based DNA delivery to 293/KDR cells proceeds mostly via a KDR/flk-1 receptor-mediated pathway. DNA/PEI-SP-FVEGF121 complexes were added to 293/KDR and 293 cells at final concentrations of 57 nM FVEGF121, 32 nM PEI-SP conjugate and 0.5 μg/well DNA (the N/P ratio of 7.5). After incubation at 4° C., cells were washed once with 0.4 M NaCl prior to incubation at 37° C. After a 24-hour incubation at 37° C. cells were lysed and luciferase expression in cell lysates was measured using a commercially available Dual-Luciferase Reporter Assay System (Promega, USA) according to the manufacturer's instructions.

### DETAILED DESCRIPTION OF THE INVENTION

As indicated above, in one aspect, the present invention is a molecular delivery vehicle for delivery of therapeutic, diagnostic, or research compounds to a target. The molecular delivery vehicle includes (a) a carrier for carrying the compounds; (b) an adapter covalently linked to the carrier; and (c) a targeting protein comprising a recognition portion and a targeting portion, the recognition portion capable of binding to the adapter, the targeting portion capable of binding to the target. The target may exist freely in an aqueous medium, or may be attached onto a natural or artificial surface.

The invention is also directed to methods of delivering the above molecular delivery vehicles, for example administering a pharmaceutical composition containing the above molecular delivery vehicles.

The present invention is also directed to a composition to coat artificial surfaces in medical devices and biosensors with a desired fusion targeting protein. In use, the invention contemplates depositing the adapter portion onto a selected surface, wherein the adapter portion serves as a target that interacts with a recognition peptide in the targeting protein.

The present invention also relates generally to methods, compounds and compositions useful for target-mediated delivery to a target (e.g., targeted cells) of therapeutic, diagnostic, or research compounds associated with a carrier conjugated to S-protein fragment of ribonuclease A that is associated with a specific S-peptide fragment of ribonuclease A fused to the targeting protein that interacts with a receptor on the target.

Another object of the present invention is to provide a method for target-mediated delivery of therapeutic, diagnostic, or research compounds which comprises contacting the cell with a molecular delivery vehicle comprising a carrier conjugated to S-protein fragment of ribonuclease A that is associated with a S-peptide fragment of ribonuclease A fused to the targeting protein that interacts with a receptor on the target.

Another object of the invention is to provide a complex comprising a carrier conjugated to S-protein fragment of ribonuclease A that is associated with S-peptide fragment of ribonuclease A fused to the targeting protein that interacts with a cell surface receptor on the target.

Still another object of the invention is to provide a molecular delivery vehicle which comprises a conjugate of S-protein fragment of ribonuclease A with suitable carrier for selected compounds that are useful for therapeutic, diagnostic, or research use.

Yet another object of the invention is to provide DNA sequence, recombinant expression vector, expression host, and a fusion protein which comprise S-peptide fragment of ribonuclease A and a targeting protein that interact with a cell surface receptor on the target.

Yet another object of the invention is to provide DNA sequence, recombinant expression vector, expression host, and a recombinant protein which comprise various forms of S-protein fragment of human or bovine ribonuclease A that interact with S-peptide fragment of human or bovine ribonuclease A.

Yet another object of the invention is to provide DNA sequence, recombinant expression vector, expression host, and a fusion protein which comprise S-protein fragment of ribonuclease A and a targeting protein that interact with a cell surface receptor on the target.

Definitions

Unless defined otherwise, all technical and scientific terms used herein have the same meaning as is commonly understood by one of skill in the art to which the subject matter herein belongs.

As used herein, “delivery” of molecular delivery vehicles to a target refers to contacting the target with the molecular delivery vehicle. In some embodiments it may be advantageous to utilize receptor-mediated endocytosis in order to achieve intracellular delivery of the molecular delivery vehicle.

As used herein “targeting protein” refers to a molecule capable of recognizing a target in an aqueous medium, or on a natural or artificial surface. The proteins of the targeting protein include cytokines, growth factors, peptide hormones, antibodies to cell surface antigens, and the like. It is to be understood that the proteins may be used as modified by amino acid substitutions, amino acid deletions, amino acid insertions, and amino acid additions, as in fusion proteins, that do not eliminate the targeting ability of the proteins.

As used herein “targets on the natural or artificial surfaces” refers to components of the natural or artificial surfaces that are capable of binding specific molecules. The targets include cell surface receptors, cell surface antigens, proteins on the surface of medical devices and biosensors, and the like.

As used herein “therapeutic, diagnostic, or research compounds” refers to any matter that is advantageously delivered to targets on the natural or artificial surfaces. The compounds may include therapeutic, diagnostic, or research compounds, and may be selected from, but not limited to DNA, RNA, chemically modified DNA, chemically modified RNA, proteins, peptides, chemically modified proteins, chemically modified peptides, paramagnetic agents, radioactive compounds, various viruses, viral and bacteriophage particles, fluorogenic agents, liposomes of various composition, and various natural and synthetic compounds.

As used herein “adapter” refers to natural or man-made entities including recombinant proteins and chemical entities that can bind a specific peptide wherein the peptides are either natural or selected from combinatorial peptide libraries. Examples of useful adapters include proteins or peptides such as either S-protein or S-peptide fragment of ribonuclease A. Additional materials that may serve as useful adaptors include S-protein fragment of bovine pancreatic ribonuclease A, cellulose, calmodulin, and streptavidin.

As used herein “recognition peptide” refers to a natural or synthetic peptide selected from a combinatorial peptide library wherein the peptide can bind to adapter and can be introduced into fusion protein by methods of recombinant DNA technology.

As used herein “fusion protein” refers to a recombinant protein that contains two or more polypeptide fragments that are encoded by DNA sequences that have been combined with the methods of recombinant DNA technology in a form that allows expression of the fusion protein in suitable hosts.

As used herein interchangeably, “S-peptide fragment of ribonuclease”, “S-peptide”, or “S-tag” refers to a 15 amino acid fragment of bovine pancreatic ribonuclease A with the following amino acid sequence: Lys-Glu-Thr-Ala-Ala-Ala-Lys-Phe-Glu-Arg-Gln-His-Met-Asp-Ser (SEQ ID NO:1), or to a 15 amino acid fragment of human ribonuclease A with the following amino acid sequence: Lys-Glu-Ser-Arg-Ala-Lys-Lys-Phe-Gln-Arg-Gln-His-Met-Asp-Ser (SEQ ID NO:2), that can associate with the S-protein fragment of ribonuclease A forming the associate known as ribonuclease S for bovine protein. It is understood that the S-peptide may be used as modified by amino acid substitutions, amino acid deletions, amino acid insertions, and amino acid additions, as in fusion protein that do not eliminate the ability of the peptide to bind to S-protein fragment of ribonuclease. Such operational definition of S-peptide encompasses peptide fragments from ribonucleases of other species that may bind to appropriate protein fragment. Vectors for the expression of fusion proteins with bovine S-tag are commercially available. Vectors for the expression of fusion proteins with human S-tag are constructed using techniques known in the art by insertion of DNA sequence encoding the human S-tag with the composition Lys-Glu-Ser-Arg-Ala-Lys-Lys-Phe-Gln-Arg-Gln-His-Met-Asp-Ser (SEQ ID NO:2) in the commercially available expression vectors.

As used herein interchangeably, “S-protein fragment of ribonuclease”, or “S-protein”, refers to a fragment of ribonuclease A that can associate with the S-peptide fragment of ribonuclease A. The natural bovine S-protein (residues 21-124) is formed after limited digestion of bovine pancreatic ribonuclease A by protease subtilisin. In the following disclosure the present invention provides for novel recombinant bovine and human S-proteins. It is to be understood that the S-protein may be used as modified by amino acid substitutions, amino acid deletions, amino acid insertions, amino acid additions, as in fusion proteins, chemical modifications, and derivatization with various low and high molecular weight reagents that do not eliminate the ability of the S-protein to bind to S-peptide fragment of ribonuclease. Such operational definition of S-protein encompasses protein fragments from ribonucleases of other species that may bind the appropriate peptide fragment. The natural bovine S-protein and some derivatives of thereof are available commercially.

As used herein, “targeted cells” refers to cells in vitro or in vivo that express components of the cell surface that are capable of binding targeting molecules.

As used herein, “carrier” for therapeutic, diagnostic, or research compounds refers to natural or synthetic molecules or aggregates thereof which can be associated covalently or non-covalently with therapeutic, diagnostic, or research compounds. Such carriers include, but are not limited to natural or synthetic polymers and co-polymers, such as dextran and other polysaccharides, polylysine, polyethylenimine, poly(vinyl alcohol), poly(divinyl) ether-co-maleic anhydrdride, poly(ethylene glycol), poly(methyl methacrylates), polyanhydrides, polyesters, polyacrylic acids, polyurethanes,N-(2-hydroxypropyl)methacrylamide, derivatized polymers and co-polymers, liposomes, various viral and bacteriophage particles, and various manufactured beads and nanoparticles.

As used herein, “S-protein linked to carrier” refers to covalent linkage of the S-protein to the carrier. In some preferred embodiments, such covalent linkage may utilize bifunctional chemical reagents that form covalent bonds with functional groups existing in the S-protein and the carrier. In other preferred embodiments the bifunctional chemical reagents may form covalent bonds with functional groups that were introduced into the S-protein and the carrier in order achieve a covalent binding of S-protein to the carrier. In some preferred embodiments, a single adapter can be linked to a single carrier, while in other preferred embodiments, more than one adapter can be linked to a single carrier in order to increase avidity of molecular delivery vehicle to the target, or in order to construct vehicle capable of binding to different targets.

As used herein, “S-protein linked to the artificial surface” refers to linkage of the S-protein to the surface. In some preferred embodiments such linkage may utilize bifunctional chemical reagents that form covalent bonds with functional groups existing in the S-protein and the surface. In other preferred embodiments the bifunctional chemical reagents may form covalent bonds with functional groups that were introduced into the S-protein and the surface in order achieve a covalent binding of S-protein to the surface.

The targeting component of the invention includes structures that generally bind other molecules, such as receptors or antigenic determinants on the surface of any targeted cell (e.g., endothelial cells, tumor cells, and the like). The targeting component may also include adapters or other artificial or biological structures that are deposited on the artificial surfaces of medical devices, biosensors, various carriers capable of linking more than one adapter and biological entities such as cells, viruses, and bacteria, expressing adapters on their surfaces. Adapters can be selected from entities known in the art and capable of association with recognition peptides, wherein the peptides are either natural or selected from combinatorial peptide libraries. In one embodiment, when adapter entity is a protein it may be incorporated into other proteins, for example viral and bacteriophage surface proteins, or into suitable targeting proteins for interaction with other entities carrying appropriate recognition peptides, by recombinant DNA methods known in the art.

The carrier component of the invention can be selected from carriers known in the art, such as but not limited to such as dextran and other polysaccharides, polyethylenimine, poly(vinyl alcohol), poly(divinyl) ether-co-maleic anhydrdride, poly(ethylene glycol), poly(methyl methacrylates), polyanhydrides, polyesters, polyacrylic acids, polyurethanes,N-(2-hydroxypropyl)methacrylamide, various liposomes and derivatized liposomes, various dendrimers and derivatized dendrimers, various viral and bacteriophage particles, various manufactured bids and nanoparticles.

The compound component of the invention can be selected from therapeutic, diagnostic, or research compounds whose functions can be established in the art, such as but not limited to nucleic acids, peptides, proteins, viruses, viral, and bacteriophage particles employed for gene delivery, chemotherapeutic agents, paramagnetic, radioactive or fluorogenic agents.

The targeting protein of the invention contains the recognition portion fused to the N-terminus or to the C-terminus of the protein or inserted in the main body of the proteins in ways that do not eliminate ability of targeting protein to bind to a target. The targeting proteins can be produced as fusion proteins by methods known in the art and can be selected from proteins known in the art but not limited to cytokines, growth factors, peptide hormones, antibodies. It is also to be understood than addition of a recognition portion to any protein protein allows it to be associated with natural or synthetic entity recognizable by said recognition portion.

The recognition portion can be selected from peptides either known in the art for their ability to bind to a chosen adapter entity, or to be selected from combinatorial peptide libraries for their ability to bind to a chosen adapter entity.

In one preferred embodiment, the present invention is directed to a molecular delivery vehicle comprising a carrier conjugated to S-protein fragment of ribonuclease A that is associated with a S-peptide fragment of ribonuclease A fused to the N-terminus of targeting protein that interacts with a cell surface receptor on the targeted cells.

According to a preferred embodiment the molecular vehicle for drug delivery comprises a liposome carrier loaded with a drug and in the following disclosure the present invention provides a method for association of liposomes with S-protein fragment of ribonuclease A.

According to another preferred embodiment the molecular vehicle for drug delivery comprises polyethylenimine as a carrier for nucleic acid and in the following disclosure the present invention provides a method for conjugation of polyethylenimine to S-protein fragment of ribonuclease A.

According to a particularly preferred embodiment the targeting protein comprises S-peptide fragment of ribonuclease A fused to the N-terminus of an isoform of vascular endothelial growth via peptide spacer and in the following disclosure the present invention provides for DNA sequences encoding those targeting proteins, recombinant expression vectors for expression of the targeting proteins and expression host.

According to another preferred embodiment the adapter is a recombinant S-protein and in the following disclosure the present invention provides for DNA sequences, expression vectors, expression host, and recombinant proteins for several human and bovine S-proteins.

The Method

As indicated above, the present invention comprises a method useful for target-mediated delivery to targeted cells of therapeutic, diagnostic, or research compounds to a target on a natural or artificial surface, or free in an aqueous medium. The present invention also comprises a method useful for immobilization of proteins on the artificial surface, included but not limited to, the surfaces of medical devices, biosensors, and high throughput screening devices. Current methods known in the art for delivery of therapeutic, diagnostic, or research compounds, or for immobilization of proteins on artificial surfaces, are based on chemical modifications of groups in the targeting protein. The chemical modifications may include, but are not limited to a direct covalent crosslinking of the entities to targeting molecules, or covalent crosslinking of a carrier of the entities to targeting molecules, or covalent crosslinking of groups (e.g. biotin) capable of capturing either carriers of the entities or the entities modified by methods known in the art. Current methods known in the art for immobilization of proteins on the artificial surface are based on chemical modifications of groups in the protein. The chemical modifications may be used to immobilize proteins by crosslinking them directly to artificial surfaces, or to groups that interact either with artificial surfaces, or with other groups deposited on the artificial surfaces by methods known in the art.

The chemical modifications of proteins have to be custom-developed on a case-by-case basis. Furthermore, the chemical modifications are always limited by the harm they may inflict upon the functional activity of the proteins. Finally, the approach based on customized chemical modification of different proteins does not allow manufacturing of standardized preparations of therapeutic, diagnostic or research compounds that can be combined with different proteins, or manufacturing of standardized artificial surfaces for immobilization of different proteins.

To overcome these and other obstacles the present invention discloses the method useful for target-mediated delivery of therapeutic, diagnostic, or research compounds, and for immobilization of different proteins on artificial surfaces. The method is based on conversion of a protein into a fusion protein that comprises a recognition peptide fused via a spacer peptide to the N-terminus or C-terminus of a targeting protein, or inserted in the main body of a targeting protein. Recognition peptide can be also expressed on the surface of viral and bacteriophage particles as part of the surface proteins, such as fiber or penton base coat proteins of adenovirus, 4070A (amphotropic) retroviral envelope protein, or protein III of bacteriophage. By definition, in order to be useful such fusion proteins should retain its functional activities. A recognition peptide has to display the ability to bind an adapter entity and may be a natural peptide or derived from combinatorial peptide libraries. Recognition peptides are known and used in the art as tags for purification of recombinant fusion proteins. Examples of natural peptides are: a 15-mer S-peptide fragment of bovine pancreatic ribonuclease A, termed S-tag, that binds the S-protein fragment of bovine pancreatic ribonuclease A (Kim & Raines, 1993), cellulose binding domains (107 to 154 aa) derived from bacterial cellulases that bind cellulose, and a 4 kDa calmodulin-binding peptide that binds calmodulin. An example of a peptide selected from a combinatorial peptide library is a peptide with intrinsic streptavidin-binding activity, termed strep-tag (Schmidt & Skerra, 1993).

An adapter entity that can bind to a recognition peptide in the fusion protein may be purified from its natural host or produced by methods of recombinant DNA technology or by chemical synthesis. An adapter entity can be chemically conjugated to a suitable carrier for a chosen therapeutic, diagnostic, or research compound and then associated with any fusion targeting protein. An adapter entity can be chemically conjugated to an artificial surface of medical device or biosensor and then associated with any fusion protein containing appropriate recognition peptide. Adapter entity can be expressed on the surface of viral and bacteriophage particles as part of the surface proteins, such as fiber or penton base coat proteins of adenovirus, 4070A (amphotropic) retroviral envelope protein, or protein III of bacteriophage, and then associated with fusion targeting protein. Thus, instead of developing customized procedures for different proteins the method relies on standardized procedures for adapter entity only.

A suitable method for chemical conjugation of an adapter to different carriers or to artificial surfaces of medical devices and biosensors, or for expression of adapter protein or a peptide on the surface of viral or bacteriophage particles is chosen among many known in the art. It is understood that an adapter may be constructed to have a particularly reactive amino acid residue suitable for a conjugation with a carrier. It is understood that carrier may be used as such or already modified with therapeutic, diagnostic, or research compound. It is also understood that artificial surfaces may be used as such, or may have other chemical groups deposited on the surface by methods known in art. If necessary, the chemical modifications can be performed on complexes of adapter entity with recognition peptide followed by purification of intact complexes, followed by separation of the conjugates from complexes by methods known in the art. The latter procedure selects for conjugates that retain the ability to bind to recognition peptide. By definition, conjugates should retain the ability to interact with recognition peptide in the fusion protein. By definition adapters expressed on the surface of viral or bacteriophage particles or attached to artificial surface of medical device or biosensor should retain the ability to interact with recognition peptide in the fusion protein.

A conjugate binds to the fusion protein via interaction between adapter entity and recognition peptide and the resulting delivery complexes are employed for target-mediated delivery. It is understood that therapeutic, diagnostic, or research compound may be loaded on a conjugate before or after binding of the conjugate to the fusion protein. By definition, in order to be useful such complexes should retain the ability to bind to targets on natural or artificial surfaces. Thus, instead of customizing procedures for loading therapeutic, diagnostic, or research compounds on different proteins the method relies on the same standardized preparation of conjugate that can be combined with different fusion proteins capable of targeting different targets on natural and artificial surfaces.

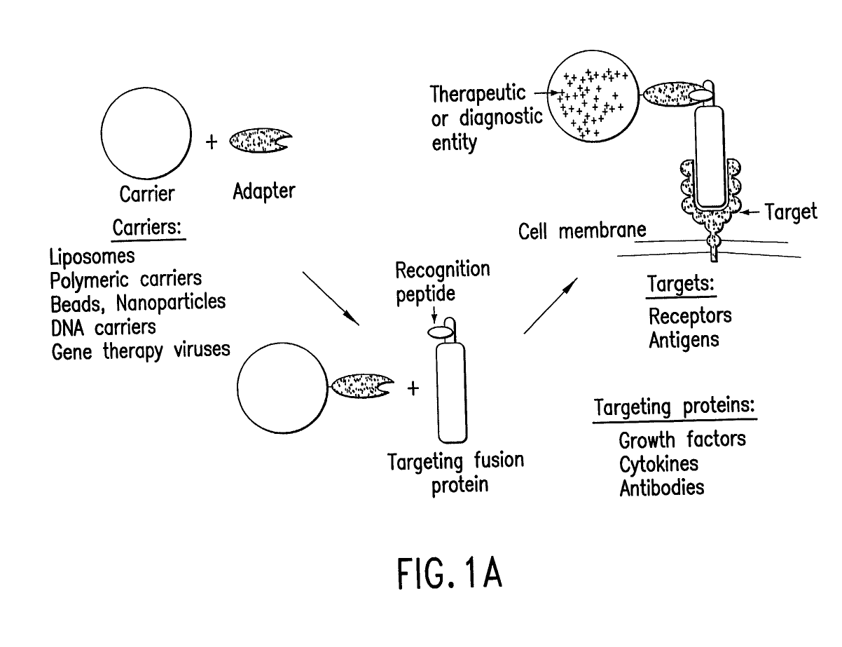

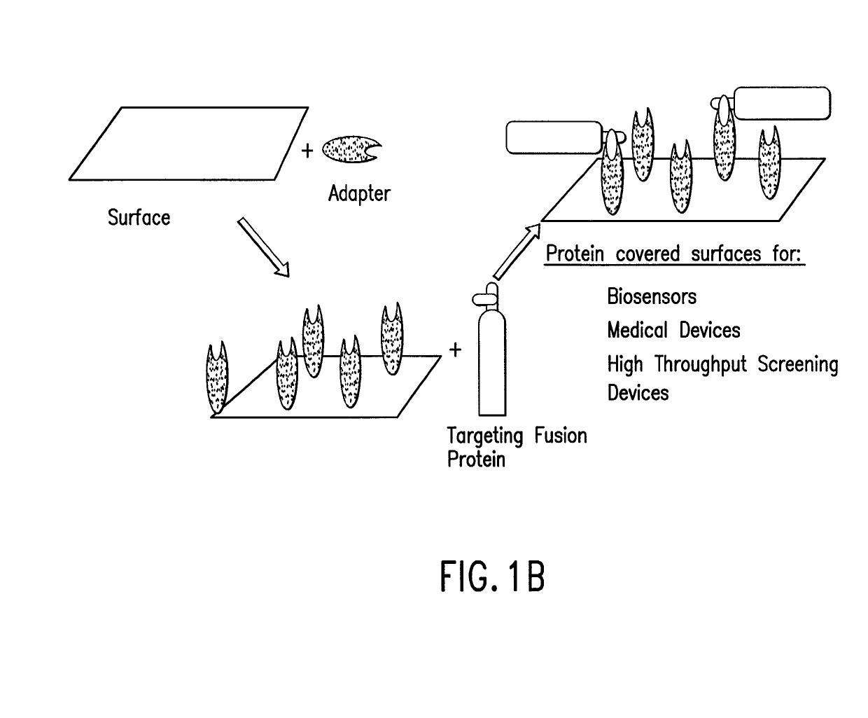

Schematic representation of the proposed method for target-mediated delivery of a therapeutic, diagnostic, or research compounds to targeted cells or to artificial surfaces is shown in FIGS. 1A and 1B.

The present invention discloses a particularly preferred embodiment of the method useful for target-mediated delivery of a therapeutic, diagnostic, or research compounds to targeted cells. The particularly preferred embodiment is based on a fusion targeting protein, which comprises an S-peptide fragment of ribonuclease A linked via a spacer peptide to the targeting protein. The S-peptide comprises only 15 amino acid residues in human or bovine ribonuclease A and binds to an S-protein that comprises only 108 and 104 amino acid residues for human and bovine proteins, respectively. The relatively small size of S-peptide/S-protein complex, known as ribonuclease S, decreases the likelihood of creating spatial constrains for interactions between targeting fusion proteins and a target on natural or artificial surfaces. Small sizes of S-protein and S-peptide also decreases the likelihood of disruption of viral or bacteriophage particle assembly when S-protein or S-peptide is fused to viral or bacteriophage surface protein. Furthermore, ribonuclease activity of ribonuclease S that arises after binding of S-protein to S-peptide in fusion targeting proteins can be readily quantitated with a commercially available kit (Novagen, USA).

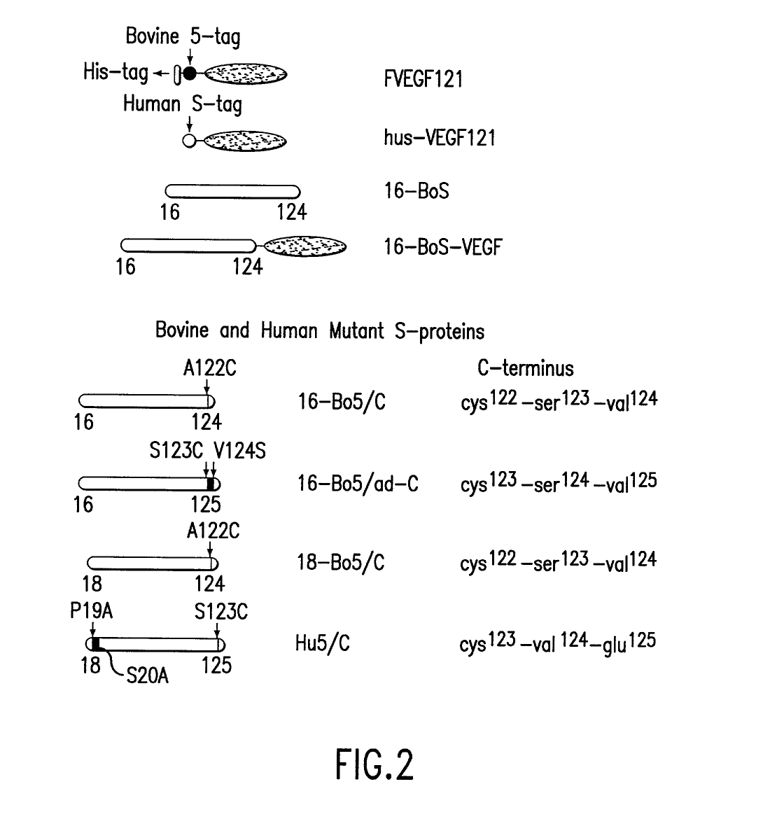

The particularly preferred embodiment is based on fusion targeting proteins, termed FVEGF, that contain vascular endothelial growth factor (FIG. 2). Vascular endothelial growth factor controls growth of endothelial cells via interaction with several receptors, among which KDR/flk-1 receptor expression is limited mostly to endothelial cells. In adult organisms the growth of endothelial cells (angiogenesis) occurs, with the exception of corpus luteum development, only in various pathological conditions. Thus, KDR/flk-1 receptor-mediated delivery of therapeutic, diagnostic, or research compounds may be useful in therapies for various pathologies. The present invention discloses that the FVEGF fusion targeting proteins are capable of binding to vascular endothelial growth factor receptors KDR/flk-1 expressed on the surface of targeted cells.

The present invention discloses a particularly preferred embodiment for production of novel recombinant human and bovine S-protein. For this and other purposes, human and bovine S-proteins are preferably engineered to be expressed as recombinant proteins that can be used as adapters, including adapters with an additional cysteine that is not involved in disulphide bonds and therefore is particulary suitable for crosslinking to the carrier (FIG. 2C).

A particularly preferred embodiment of the present invention is a method useful for target-mediated delivery of drug-loaded liposomes to targeted cells expressing KDR/flk-1 receptors. For these and other purposes, the S-protein fragment of bovine ribonuclease A, used as an adapter protein, is chemically conjugated to polyethylene glycolated phospholipid to achieve association of S-protein with liposome in such a way that the conjugate and associate between conjugate and liposome retain the ability to bind to the S-peptide fragment of bovine ribonuclease A. A drug-loaded liposome/S-protein construct (termed Lip(Dox)-SP) is associated with FVEGF fusion targeting protein containing S-peptide fragment of ribonuclease A and the resulting complex is employed for KDR/flk-1 receptor-mediated liposome delivery.

The present invention also discloses a particularly preferred embodiment of the method useful for target-mediated delivery of DNA to targeted cells expressing KDR/flk-1 receptors. For this and other purposes the S-protein fragment of bovine pancreatic ribonuclease A, used as an adapter protein, is chemically conjugated to polyethylenimine in such a way that the conjugate retains the ability to bind to the S-peptide fragment of bovine pancreatic ribonuclease A. The conjugate (termed PEI-SP) is associated with FVEGF fusion targeting protein containing S-peptide fragment of bovine pancreatic ribonuclease A and the resulting complex is associated with DNA for KDR/flk-1 receptor-mediated DNA delivery.

The Composition

1. Fusion Targeting Proteins.

The present invention comprises a composition useful for target-mediated delivery of a therapeutic, diagnostic, or research compound to natural and artificial surfaces. One element of this composition is a fusion targeting protein which comprises a recognition peptide fused via a spacer peptide to the N-terminus or C-terminus or inserted in the main body of a targeting protein. A targeting protein is selected from protein molecules capable of recognizing a component on the natural or artificial surface. Also, by definition, every fusion protein containing recognition peptide is capable of recognizing adapter protein. Known targeting molecules include, but are not limited to cytokines, growth factors and antibodies against cell surface antigens. It is understood that a targeting protein may be used in the proposed composition if the protein can be expressed as part of the recombinant fusion protein using recombinant DNA technology known in the art and if the recombinant fusion protein retains the ability to bind to the target on the natural or artificial surface. Targeting proteins are selected to deliver therapeutic, diagnostic, or research compounds exclusively, or preferentially to designated targets on natural or artificial surface.

It is known in the art that many proteins can be expressed as functionally active fusion proteins with an additional peptide fused to their N-termini or C-termini. Using commercially available plasmids, recombinant proteins are customarily expressed with peptide tags, such as but not limited to myc-tag, S-tag, cellulose-binding tag, calmodulin-binding tag, or strep-tag that facilitate detection and purification of the proteins. In general, a tag may be a part of a known protein, or be selected from combinatorial peptide libraries.

The present invention discloses a particularly preferred embodiment of the composition useful for target-mediated delivery to targeted cells of a therapeutic, diagnostic, or research compound. The particularly preferred embodiment is based on a fusion targeting protein, termed FVEGF, which comprises an S-peptide fragment of ribonuclease A linked via a spacer peptide to vascular endothelial growth factor (FIG. 2). The present invention discloses that the FVEGF fusion targeting protein is capable of binding to vascular endothelial growth factor receptors KDR/flk-1 expressed on the surface of targeted cells.

2. Conjugate of a Carrier and an Adapter Protein

Another element of the composition disclosed in the invention is a conjugate of a carrier and an adapter entity. It is known in the art that many carriers may be used for delivery of therapeutic, diagnostic, or research compounds. Such carriers include but are not limited to natural or synthetic polymers and co-polymers, such as dextran and other polysaccharides, polylysine, polyethylenimine, poly(vinyl alcohol), poly(divinyl) ether-co-maleic anhydrdride, poly(ethylene glycol), poly(methyl methacrylates), polyanhydrides, polyesters, polyacrylic acids, polyurethanes,N-(2-hydroxypropyl)methacrylamide, derivatized polymers and co-polymers, liposomes, various viral and bacteriophage particles, and various manufactured beads and nanoparticles.

An adapter entity may be purified from its natural host or produced by methods of recombinant DNA technology or by chemical synthesis. It is understood that different proteins may serve as adapter entities if recognition peptides to which they can bind, either exist in nature or are selected from combinatorial peptide libraries. Also, natural or synthetic polymers that can bind to recognition peptides can serve as adapter entities. A suitable method of conjugation of carriers to adapter entities is chosen among many known in the art. These methods include, but are not limited to chemical conjugation. It is understood that an adapter may be constructed to have a particularly reactive amino acid residue suitable for a conjugation with a carrier. It is also understood that the carrier may be used as such or already modified with therapeutic, diagnostic, or research compounds. If necessary, the conjugation can be performed on complexes of adapter entity with recognition peptide followed by purification of intact complexes, followed by purification of the conjugates from complexes by methods known in the art. The latter procedure selects for conjugates that retain the ability to bind to recognition peptides.

The present invention discloses a particularly preferred embodiment of the conjugates useful for target-mediated delivery to targeted cells of liposomes loaded with therapeutic or diagnostic agents and conjugates useful for target-mediated delivery to targeted cells of DNA and other nucleic acids and chemically modified nucleic acids. The particularly preferred conjugates comprise derivatized phospholipid conjugated to S-protein fragment of ribonuclease A for association with liposome and polyethylenimine conjugated to S-protein fragment of ribonuclease A. The present invention discloses also a particularly preferred embodiment for recombinant human and bovine S-proteins for developing new adapter-carrier conjugates.

3. Conjugate of an Adapter Protein and Artificial Surface

Another element of the composition disclosed in the invention is a conjugate of an adapter entity and artificial surface. It is known in the art that many artificial surfaces have to be covered with proteins in order to be used. Such artificial surfaces include but are not limited to artificial surfaces of biosensors, medical devices, implantable slow release constructs for controlled drug release, and surfaces for immobilization of proteins for a high throughput screening.

An adapter entity may be purified from its natural host or produced by methods of recombinant DNA technology or by chemical synthesis. It is understood that different proteins may serve as adapter entities if recognition peptides to which they can bind, either exist in nature or are selected from combinatorial peptide libraries. Also, natural or synthetic polymers that can bind to recognition peptides can serve as adapter entities. A suitable method of conjugation of adapter entities to artificial surfaces is chosen among many known in the art. These methods include, but are not limited to chemical conjugation of adapter entity to artificial surface. If necessary, the conjugation can be performed on complexes of adapter entity with recognition peptide followed by removal of recognition peptide from complexes by methods known in the art.

The present invention discloses a particularly preferred embodiment of the adapter entity. The particularly preferred adapter entity comprises S-protein fragment of ribonuclease A that readily conjugate, for example, to the surface of a biosensor, or a medical device, or an implantable device for controlled drug release, or a surface for immobilization of proteins for a high throughput screening and retains the ability to bind S-peptide containing biologically active proteins.

4. Fusion Proteins Containing Adapter Entity

Another element of the composition disclosed in the invention is viral or bacteriophage fusion protein containing a protein or a peptide moiety that function as an adapter entity. It is known in the art that viral and bacteriophage particles can be used for nucleic acid delivery and that certain surface proteins can be made as fusion proteins without disrupting particle's functions.

An adapter protein or peptide is inserted into viral or bacteriophage surface protein by methods of recombinant DNA technology chosen among many known in the art. It is understood that different viral or bacteriophage surface proteins may be used for insertion. It is understood that different proteins or peptides may serve as adapter entities if recognition entities to which they can bind, either exist in nature or are selected from combinatorial peptide libraries.

The present invention discloses particularly preferred embodiments of the adapter entity useful for insertion in viral and bacteriophage surface proteins. The particularly preferred adapter entities comprise either S-protein or S-peptide fragment of ribonuclease A.

The molecular delivery vehicle of the present invention is useful in treating pathophysiological conditions that depend on cells that can be detected or affected via target-mediated delivery of therapeutic or diagnostic compounds. Such conditions include, but are not limited to, all forms of cancer, cardiovascular diseases, neurological diseases, eye diseases, wound healing, and the like.

The molecular delivery vehicle of the present invention may be administered to a patient in the form of a pharmaceutical composition. The pharmaceutical compositions of the present invention are preferably administered internally, e.g., intravenously, in the form of conventional pharmaceutical preparations, for example in conventional enteral or parenteral pharmaceutically acceptable excipients containing organic and/or inorganic inert carriers, such as water, gelatin, lactose, starch, magnesium stearate, talc, plant oils, gums, alcohol, petroleum jellies such as Vaseline, or the like. The pharmaceutical compositions can be in conventional solid forms, for example, tablets, dragees, suppositories, capsules, or the like, or conventional liquid forms, such as suspensions, emulsions, or the like. If desired, they can be sterilized and/or contain conventional pharmaceutical adjuvants, such as preservatives, stabilizing agents, wetting agents, emulsifying agents, buffers, or salts used for the adjustment of osmotic pressure. The pharmaceutical preparations may also contain other therapeutically active materials.

A therapeutically effective dosage of a vehicle for target-mediated delivery is that dosage that provides the desired effect. In general, an effective dosage should be in the range from 0.1 ng to 100 mg of targeting protein per ml of serum. As known to those skilled in art, depending on the pharmaceutical composition and the route of administration the above range may be achieved upon administration of a dosage of targeting compound from 0.01 to 2000 mg per day per kg of patient's weight and preferably from 0.01 to 200 mg per day per kg of patient's weight, more preferably from 0.01 to 20 mg per day per kg of patient's weight.

### EXAMPLES

The following Examples are intended to illustrate, but in no way limit the scope of the present invention. All parts and percentages are by weight and all temperatures are in degrees Celsius unless explicitly stated otherwise.

### Example 1

### Preparation of Fusion Proteins Comprising S-Peptide or S-Protein Fragment of Ribonuclease Linked to Vascular Endothelial Growth Factor Via Peptide Spacer and Preparation of Recombinant S-Proteins

A. General Descriptions

1. Bacterial Strains and Plasmids

E. coli strain DH5α is commercially available from Life Technologies (USA). E. coli strain B121 (DE3)pLysS, Tuner(DE3)pLacI, Origami B(DE3)pLacI are commercially available from Novagen (USA). Vector pETBlue-1 for blunt-end cloning and vector pET32a(+) for bacterial expression of recombinant proteins with a terminal extension containing His-tag, S-tag, and thioredoxin are commercially available from Novagen (USA). Plasmid pET-HP containing synthetic DNA fragment encoding 1-127 aa of mature human pancreatic Ribonuclease A and plasmid pT7-7/RibonucleaseA containing cDNA encoding 1-124 aa of mature bovine pancreatic Ribonuclease A were obtained from Dr. G. D'Alessio (Napoli Federico II University, Naples, Italy). Plasmids pLen-121, pLen-165, and pLen-189 containing the DNA sequence encoding the 121-165- and 189-residue isoforms of human VEGF has been described in U.S. Pat. No. 5,219,739, herein incorporated by reference in it's entirety, and were obtained from Dr. J. Abraham (Scios Nova, Inc., USA). Mammalian cell expression plasmid pBal/Pst/pur-KDR containing DNA sequence encoding full length KDR/flk-1 receptor has been obtained from Dr. Terman (A. Einstein School of Medicine, USA). Reporter plasmid pRL-tk for expression of luciferase in mammalian cells is commercially available from Promega (USA).

2. DNA Manipulations

The restriction and modification enzymes employed herein are commercially available in the U.S. and were used according to the manufacturer's instructions. Preparation of competent cells, transformation, and bacterial medium were according to Sambrook et al. (J. Sambrook, E. F. Fritsch and T. Maniatis. (1989) Molecular Cloning: A Laboratory Manual, Cold Spring Harbor Laboratory Press, Cold Spring Harbor, N.Y.) or according to the manufacturer's instructions. Purification of plasmids was done using Wizard Plus SV Minipreps or Maxipreps DNA Purification Systems (Promega, USA) according to the manufacturer's instructions. The purification of DNA fragments from agarose gels was done using the Geneclean Spin kit (Bio 101, USA) according to the manufacturer's instructions.

B. Sub-Cloning of 121-, 165-, and 189-Residue Isoform of Human VEGF into apET32a(+) Expression Vector

1. Primers for Amplification of DNA Encoding 121-, 165-, and 189-Residue Isoforms of Human VEGF

Primers for human VEGF DNA amplifications were synthesized by GeneLink (USA). The primer corresponding to the “sense” strand of all isoforms of VEGF (SEQ ID NO:2) included an Stu I restriction site immediately upstream of the DNA codon for amino acid-I of the mature 121-, 165-, and 189-residue isoforms of VEGF.

The primer corresponding to the “antisense” strand of all isoforms of VEGF (SEQ ID NOS:3 and 4) complemented the coding sequence of the DNA encoding the carboxyl end of the mature peptides, including a translation stop codon after the sequence encoding mature VEGF, and introduced an Xho I restriction site downstream of the VEGF-encoding DNA and the stop codon.

| | |
|---|---|
| 5′-TAAGGCCTATGGCAGAAGGAGGAGGG-3′ | SEQ ID NO:3 |
| 5′-ACTCGAGTCACCGCCTCGGCTTGTCAC-3′ | SEQ ID NO:4 |

2. PCR to Amplify DNA Encoding 121-, 165-, and 189-residue Isoforms of Human VEGF

The human VEGF cDNA was amplified by PCR from the pLen-121, pLen-165, and pLen-189 plasmids, containing the DNA coding sequences for the 121-, 165-, and 189-residue isoforms of VEGF, respectively. Ten nanograms of each template DNA were mixed in a standard PCR reaction, containing 10 μmol of each oligonucleotide, 0.2 mM dNTP and 2 U of Pfu DNA polymerase (New England Biolabs, USA) in Pfu buffer. Incubations were done in a DNA GenAmp PCR System 2400 (Perkin Elmer Cetus, USA). One cycle included a denaturation step (94° C. for 1 min), an annealing step (65° C. for 1 min), and an elongation step (72° C. for 1 min). After 25 cycles, a 10 μl aliquot of each reaction was run on a 1% agarose gel to verify the correct size of the amplified product. The amplified DNA fragments were digested with Stu I and Xho I and purified with the Geneclean Spin kit (BIO 101, USA).

3. pET32-txVEGF121, pET32-txVEGF165, and pET32-txVEGF189 Plasmid Construction.

Amplified DNA fragments described in Example 1.B.2 were ligated into a pET32a(+) vector using the Xho I site from multiple cloning site of the vector and the Stu I site, which was constructed by treating the vector as follows: pET32a(+) DNA was linearized with Nco I restrictase, and the recessed termini were partially filled with cytidine using DNA polymerase I Large (Klenow) fragment. Then the construct was purified with the Geneclean Spin kit (BIO 101, USA) and single-stranded overhangs were removed with mung bean nuclease. The resulting construct was digested with Xho I restrictase and purified with the Geneclean Spin kit (BIO 101, USA). The ligation was accomplished such that the first amino acid of the mature 121-, 165-, and 189-residue isoforms of VEGF became the first amino acid after an enterokinase cleavage site provided by vector. The resulting plasmids were designated pET32-txVEGF121, pET32-txVEGF165, and pET32-txVEGF189, respectively, and transformed into DH5α competent cells (Life Technologies, USA) according to the manufacturer's instructions. Bacterial cultures containing the desired plasmids were grown further in order to obtain large preparations of isolated plasmids using methods described in Example 1.A.2.

4. pET32-FVEGF121, pET32-VEGF165 and pET32-VEGF189 Plasmid Construction.

The thioredoxin (tx) gene was removed from the pET32-txVEGF121, pET32-txVEGF165, and pET32-txVEGF189 plasmids by digestion of the purified plasmid DNA with restrictase Nde I followed by intramolecular ligation of the linearized plasmid DNAs with T4 ligase. The resulting plasmids were designated pET32-FVEGF121, pET32-VEGF165 and pET32-VEGF189 and transformed into DH5α competent cells (Life Technologies, USA) according to the manufacturer's instructions. Bacterial cultures containing the desired plasmids were grown further in order to obtain large preparations of isolated plasmids using methods described in Example 1.A.2.

C. Sub-Cloning of cDNA Fragments Encoding Bovine and Human S-proteins into pETBlue-1 Vector

1. Primers for Amplification of DNA Encoding 16-124, 17-124, 18-124,19-124, 20-124, 21-124 aa Fragments of Bovine Ribonuclease A

The primers corresponding to the “sense” strand of bovine S-protein cDNA (SEQ ID NOS:5, 6, 7, 8, 9, and 10) included AUG codon immediately upstream of the DNA codon for amino acid 16, 17, 18, 19, 20 and 21 of the bovine ribonuclease A, respectively.

| | |
|---|---|
| 5′-ATGAGCAGCTCCAACTACTGTAACCAG-3′ | SEQ ID NO:5 |
| 5′-ATGACTTCCGCTGCCAGCAGCTCC-3′ | SEQ ID NO:6 |
| 5′-ATGTCCGCTGCCAGCAGCTCCAAC-3′ | SEQ ID NO:7 |
| 5′-ATGGCTGCCAGCAGCTCCAACTACTG-3′ | SEQ ID NO:8 |
| 5′-ATGGCCAGCAGCTCCAACTACTGTAACC-3′ | SEQ ID NO:9 |
| 5′-ATGAGCAGCTCCAACTACTGTAACCAG-3′ | SEQ ID NO:10 |

The primer corresponding to the “antisense” strand of bovine S-protein cDNA (SEQ ID NO:11) complemented the coding sequence of the DNA encoding the carboxyl end of this protein, including a translation stop codon after the sequence encoding C-terminal ala ¹²²-ser¹²³-val¹²⁴. The primer corresponding to the “antisense” strand of bovine mutant A122C S-protein cDNA (SEQ ID NO:12) complemented the coding sequence of the DNA encoding the carboxyl end of this protein, including a translation stop codon after the sequence encoding C-terminal cys¹²²-ser¹²³-val¹²⁴. The primer corresponding to the “antisense” strand of bovine mutant S123C, V124S S-protein cDNA (SEQ ID NO:13) complemented the coding sequence of the DNA encoding the carboxyl end of this protein, including a translation stop codon after the sequence encoding C-terminal ala¹²²-cys¹²³-ser¹²⁴-val¹²⁵.

| | |
|---|---|
| 5′-CTACACTGAAGCATCAAAGTGGACTGGC-3′ | SEQ ID NO:11 |
| 5′-CTACACTGAACAATCAAAGTGGACTGGC-3′ | SEQ ID NO:12 |
| SEQ ID NO:13 | |
| 5′-CTACACTGAGCAAGCATCAAAGTGGACTGGCACG-3′ | |

2. Primers for Amplification of DNA Encoding 16-127, 19-127, 21-127 aa Fragments of Human Ribonuclease A

The primers corresponding to the “sense” strand of human ribonuclease A cDNA (SEQ ID NOS: 14, 15, and 16) included AUG codon immediately upstream of the DNA codon for amino acid 16, 19, and 21 of the human ribonuclease A, respectively.

| | |
|---|---|
| 5′-ATGGACTCGAGCCCGTCTTCTTCTTC-3′ | SEQ ID NO:14 |
| 5′-ATGGCTGCCAGCAGCTCCAACTACTG-3′ | SEQ ID NO:15 |
| 5′-ATGTCTTCTTCTACGTACTGCAACCAG-3′ | SEQ ID NO:16 |

The primer corresponding to the “antisense” strand of human S-protein cDNA (SEQ ID NO:17) complemented the coding sequence of the DNA encoding the carboxyl end of this protein, including a translation stop codon after the sequence encoding C-terminal val¹²⁴-glu¹²⁵-asp¹²⁶-ser¹²⁷. The primer corresponding to the “antisense” strand of human mutant VI22C S-protein cDNA (SEQ ID NO: 18) complemented the coding sequence of the DNA encoding the carboxyl end of this protein, including a translation stop codon after the sequence encoding C-terminal cys¹²⁴-glu¹²⁵-asp¹²⁶-ser¹²⁷. The primer corresponding to the “antisense” strand of human mutant,D126C,S127D,S128 S-protein cDNA (SEQ ID NO:19) complemented the coding sequence of the DNA encoding the carboxyl end of this protein, including a translation stop codon after the sequence encoding C-terminal val¹²⁴-glu¹²⁵-cys¹²⁶-asp¹²⁷-ser¹²⁸.

| | |
|---|---|
| 5′-TCAAGAGTCTTCAACAGACGCGTCG-3′ | SEQ ID NO:17 |
| 5′-TCAAGAGTCTTCACAAGACGCGTCG-3′ | SEQ ID NO:18 |
| SEQ ID NO:19 | |
| 5′-TCAAGAGTCGCATTCAACAGACGCGTCGAAATG-3′ | |

3. Primers for Amplification of DNA Encoding 18-125 aa Mutant Human S-protein

The primer corresponding to the “sense” strand of human mutant P19A, S20A S-protein cDNA (SEQ ID NO:20) included AUG codon immediately upstream of the sequence encoding N-terminal ser¹⁸-ala¹⁹-ala²⁰-ser²¹-ser²²-ser²³. The primer corresponding to the “sense” strand of human mutant P19A, S20A S-protein cDNA (SEQ ID NO:21) included AUG codon immediately upstream of the sequence encoding N-terminal ser¹⁸-ala¹⁹-ala²⁰-ser²¹-ser²²-ser²³ and contained codons that encode ser¹⁸, ser21, and ser²² in bovine S-protein. The primer corresponding to the “sense” strand of human mutant P19A, S20A,T24D S-protein cDNA (SEQ ID NO:22) included AUG codon immediately upstream of the sequence encoding N-terminal ser¹⁸-ala¹⁹-ala²⁰-ser21-ser²²-ser²³ and contained codons that encode ser¹⁸, ser²¹, ser²², ser²³, and asp²⁴ in bovine S-protein.

| | |
|---|---|
| 5′-ATGAGCGCTGCCTCTTCTTCTACGTACTGCAACC | SEQ ID NO:20 |
| AG-3′ | |
| 5′-ATGTCCGCTGCCAGCAGCTCTACGTACTGCAACC | SEQ ID NO:21 |
| AGATG-3′ | |
| 5′-ATGTCCGCTGCCAGCAGCTCCAACTACTGCAACC | SEQ ID NO:22 |
| AGATGATGCGTCG-3′ | |

The primer corresponding to the “antisense” strand of human S-protein cDNA (SEQ ID NO:23) complemented the coding sequence of the DNA encoding the carboxyl end of this protein, including a translation stop codon after the sequence encoding C-terminal ser¹²³-val¹²⁴-glu¹²⁵. The primer corresponding to the “antisense” strand of human mutant S123C S-protein cDNA (SEQ ID NO:24) complemented the coding sequence of the DNA encoding the carboxyl end of this protein, including a translation stop codon after the sequence encoding C-terminal cys¹²³-val¹²⁴-glu¹²⁵.

| | |
|---|---|
| 5′-TCATTCAACAGACGCGTCGAAATGAACCGG-3′ | SEQ ID NO:23 |
| 5′-CTATTCAACACACGCGTCGAAATGAACCGG-3′ | SEQ ID NO:24 |

4. Construction of Plasmids for Expression of Human and Bovine S-proteins

cDNAs corresponding to wild-type and mutant forms of human and bovine S-proteins were amplified by PCR from the pET-HP and pT7-7/RNaseA plasmids, respectively. Amplification cycles were as follows: one cycle included 94° C. for 30 sec, 58° C. for 30 sec, 72° C. for 30 sec. After 25 cycles, a 10-μl aliquot of each reaction was run on a 1% agarose gel to verify the correct sizes of the amplified products. The amplified DNA fragments were gel-purified from 1% agarose and cloned into pETBlue-1 plasmid using Perfectly Blunt Cloning Kit (Novagen, USA) according to the manufacturer's instructions. The resulting plasmids were transformed into NovaBlue competent cells (Novagen, USA) according to the manufacturer's instructions. Bacterial cultures containing the desired plasmids were grown further in order to obtain large preparations of isolated plasmids using methods described in Example 1.A.2.

D. Sub-Cloning of cDNA Fragments Corresponding to Bovine S-protein into pET/VEGF121 Vector in Frame with VEGF

1. Primers for Amplification of DNA Encoding 16-124 aa Fragment of Bovine Ribonuclease A for Construction of Bovine S-protein/VEGF121 Fusion Protein

The primer corresponding to the “sense” strand of bovine S-protein included an Nde I restriction site immediately upstream of the DNA codon for ser¹⁶ of this protein (SEQ ID NO:25). The primer corresponding to the “antisense” strand of bovine S-protein cDNA (SEQ ID NO:26) complemented the coding sequence of the DNA encoding the carboxyl end of this protein including Kpn I restriction site after C-terminal val¹²⁴.

SEQ ID NO:25
5′-AACATGCATATGAGCACTTCCGCTGCCAGCAGC-3′
SEQ ID NO:26
5′-TACGGTACCCACTGAAGCATCAAAGTGGACTGGC-3′

2. PCR to Amplify DNA Bovine S-protein for Construction of Bovine S-protein/VEGF121 Fusion Protein

cDNA corresponding to 16-124 aa fragment of bovine Ribonuclease A was amplified by PCR from the pT7-7/RibonucleaseA plasmid, using SEQ ID NOS:25 and 26. Amplification cycles were as follows: one cycle included 94° C. for 30 sec, 58° C. for 30 sec, 72° C. for 30 sec. After 25 cycles, a 10-μl aliquot of the reaction was run on a 1% agarose gel to verify the correct size of the amplified product. The amplified DNA fragments were digested with Nde I and Kpn I and gel-purified from 1% agarose.

3. pET/BoS-VEGF Plasmid Construction of pET/BoS-VEGF Plasmid.

Amplified DNA fragment described in Example 1.D.2 was ligated into a pET/VEGF121 vector using the Nde I and the Kpn I sites of the vector. The resulting plasmid was designated pET/BoS-VEGF121, and transformed into NovaBlue competent cells (Novagen, USA) according to the manufacturer's instructions. Bacterial cultures containing the desired plasmids were grown further in order to obtain large preparations of isolated plasmids using methods described in Example 1.A.2.

E. Construction of pET/hus-VEGF Plasmid Encoding FVEGF with N-Terminal Human S-Tag.

A single-stranded DNA fragment (SEQ ID NO:27) corresponding to the “sense” strand of human ribonuclease A cDNA encoding S-peptide Lys-Glu-Ser-Arg-Ala-Lys-Lys-Phe-Gln-Arg-Gln-His-Met-Asp-Ser (SEQ ID NO:28) and a complementary single-strand DNA fragment (SEQ ID NO:29) were mixed at equimolar concentrations and annealed at room temperature for 10 min. The fragments were designed to reconstitute 5′-Nde I site upstream of K1 codon and 3′-Kpn I site downstream of S15 codon after annealing. The annealed DNA fragment was ligated into a pET/VEGF121 vector between Nde I and the Kpn I sites. The resulted plasmid was designated pET/hus-VEGF121, and transformed into NovaBlue competent cells (Novagen, USA) according to the manufacturer's instructions. Bacterial cultures containing the desired plasmids were grown further in order to obtain large preparations of isolated plasmids using methods described in Example 1.A.2.

| | |
|---|---|
| 5′-ATGAAAGAATCTAGACGTAAAAAATTTCAACGTCAACACATGGACTCTGGTAC-3′: | SEQ ID NO:27 |
| 5′-CAGAGTCCATGTGTTGACGTTGAAATTTTTTACGTCTAGATTCTTTCA-3′: | SEQ ID NO:29 |

F. Expression and Purification of Recombinant VEGF Fusion Proteins and Recombinant S-Proteins.

1. Expression of VEGF Fusion Proteins in E. coli BL21(DE3)pLysS.

The pET32-txVEGF121, pET32-txVEGF165, pET32-txVEGF189, pET32-VEGF121, pET32-VEGF165, pET32-VEGF189, pET/hus-VEGF121 and pET/BoS-VEGF transformed E. coli cells BL21(DE3)pLysS were grown under conditions in which the expression of the VEGF proteins is repressed by the lac repressor to an optical density (OD) in or at the middle of the log phase of growth after which IPTG was added to induce expression of the VEGF-encoding DNA.

To generate a large-batch culture of pET32-txVEGF121, pET32-txVEGF165, pET32-txVEGF189, pET32-VEGF121, pET32-VEGF165, pET32-VEGF189, pET/hus-VEGF121 and pET/BoS-VEGF transformed E. coli cells, overnight cultures (lasting approximately 16 hours) of the transformed cells were grown in LB broth (see eg., Sambrook et al. (1989) Molecular Cloning: A Laboratory Manual, Cold Spring Harbor Laboratory Press, Cold Spring Harbor, N.Y.) containing 50 mg/L ampicillin and 34 mg/L chloramphenicol. Each of the overnight cultures was diluted 1:100 into a flask containing 100 ml LB broth with 50 mg/L ampicillin and 34 mg/L chloramphenicol and grown shaking at 37° C. until the optical density at 600 nm reached 0.5 measured in a spectrophotometer (Ultraspec 1000, Pharmacia Biotech, USA). At that stage, the expression of VEGF fusion proteins in each of the cultures was induced by the addition of isopropylthiogalactoside (IPTG) (Gibco, USA) to a final concentration of 1 mM. The induced cultures were grown for two additional hours and then harvested by centrifugation (25 min., 5000× g).

2. Purification of VEGF Fusion Proteins from Inclusion Bodies.

To purify each of VEGF fusion proteins a corresponding cell culture pellet obtained as described in Example 1.C.1 was resuspended in ice cold buffer A (50 mM Tris-HCl pH 7,5, 0.1 mM MgCl₂, 0.1 mM DTT, 200 mg/L PMSF, 25 mg/L antitripsin, 50 mg/L leupeptin, 25 mg/L aprotinin). After five cycles of freezing and thawing DNAse was added to the cell suspension, 50 U per ml. The suspension was incubated for 20 min at room temperature; then centrifuged at 5,000× g for 30 min at 4° C. The inclusion bodies pellet was solubilized in 10 ml of 8 M urea, followed by sonication for 5-10 min in an ice-cold water sonicator (FC 14, Fisher Sci., USA) and the protein solution was clarified by centrifugation at 14,000× g for 10 min, at 4° C., and the supernatant was dialyzed against 10 mM Tris-HCl pH 8.0, 150 mM NaCl for 16 hours at 4° C. VEGF fusion proteins obtained as described here were 75-90% pure as judged by Coumassie-stained SDS-PAGE analysis. VEGF fusion proteins containing thioredoxin were designated txVEGF121, txVEGF165, and txVEGF189. VEGF fusion proteins without thioredoxin were designated FVEGF121, FVEGF165 and FVEGF189. The fusion protein consisting of 16-124 aa fragment of bovine ribonuclease A linked to human VEGF121 via 7 aa linker GTDDDDK (SEQ ID NO:30) was designated 16-BoS-VEGF121. The fusion protein consisting of 1-15 aa fragment of human ribonuclease A linked to human VEGF121 via 7 aa linker GTDDDDK was designated hus-VEGF. The concentrations of VEGF fusion protein with bovine S-tag were measured with a commercially available S-tag Rapid Assay Kit (Novagen, USA) based on quantitation of ribonuclease activity which is restored when a protein carrying the S-peptide fragment of ribonuclease (S-tag) is supplemented with the S-protein fragment of ribonuclease. Concentration of hus-VEGF was determined by SDS-PAGE with FVEGF121 as a standard. Solutions of all VEGF fusion proteins were supplemented with glycerol to a final concentration of 10% v/v and stored in aliquots at −20° C. Schematic representations of the VEGF fusion proteins comprising the S-peptide or S-protein fragment of ribonuclease linked to the N-terminus of the corresponding vascular endothelial growth factor via a peptide spacer are presented in FIG. 2.

3. Expression of Recombinant Proteins Containing Fragments of Bovine ribonuclease A in E. coli Tuner(DE3)pLacI.

Clones of Tuner(DE3)pLacI bacterial cells containing plasmids described in Examples 1.C.4 were grown overnight in LB broth (Life Technologies, USA) containing 50 mg/L ampicillin and 34 mg/L chloramphenicol. Each of the overnight cultures was diluted 1:50 into a flask containing 100 ml LB broth with 50 mg/L ampicillin and 34 mg/L chloramphenicol and grown shaking at 37° C. until the optical density at 600 nm reached 0.5 measured in a spectrophotometer (Ultraspec 1000, Pharmacia Biotech, USA). At that stage, the expression of recombinant proteins in each of the cultures was induced by the addition of IPTG (Gibco, USA) to a final concentration of 1 mM. The induced cultures were grown for three additional hours and then harvested by centrifugation (25 min., 5000× g).

4. Purification of Recombinant Proteins Containing Fragments of Bovine Ribonuclease A From Inclusion Bodies.

Recombinant proteins containing 16-124 aa and 18-124 aa fragments of bovine ribonuclease A were designated 16-BoS and 18-BoS, respectively. Recombinant proteins containing 16-124 aa or 18-124 aa fragments of bovine ribonuclease A with the A112C amino acid substitutions were designated 16BoS/C and 18BoS/C, respectively. Recombinant S-proteins containing 16-125 aa or 18-125 aa fragments of bovine ribonuclease A with an additional cysteine between A122 and former S123 were designated 16-BoS/ad-C and 18-BoS/ad-C, respectively. The fusion protein consisting of 16-124 aa fragment of bovine ribonuclease A linked to human VEGF 121 via 7 aa linker GTDDDDK (SEQ ID NO:30) was designated 16-BoS-VEGF121. Recombinant proteins were stored in aliquots at −70° C. Schematic representations of some of these proteins are presented in FIG. 2.

Recombinant proteins were found in inclusion bodies that were purified from cell culture pellet using BugBuster reagent (Novagen, USA) according to the manufacturer's instructions. The purified inclusion body pellets were solubilized in of 8 M urea by sonication for 30 sec using VirSonic 475 (The Virtis Company, USA) working at 40% output power; and then dialyzed against buffer containing 10 mM Tris-HCl pH 8.0, 150 mM NaCl for 16 hours at 4° C. Recombinant proteins obtained as described here were 70-80% pure as judged by Coumassie-stained SDS-PAGE analysis.

For functional activity testing recombinant proteins were mixed with varying amounts of bovine S-peptide Lys-Glu-Thr-Ala-Ala-Ala-Lys-Phe-Glu-Arg-Gln-His-Met-Asp-Ser (SEQ ID NO:1), available from PeptidoGenic Research & Co, USA); and then added to reaction buffer containing 20 mM Tris-HCl pH 7.5, 0.1 M NaCl, 0.1 mg/ml poly(C). After 5-min incubations at room temperature reaction was stopped by 5% TCA and after 5 min incubation on ice acid-precipitable material was removed by centrifugation. Reconstituted ribonuclease activity was measured by reading the absorbance of the supernatant at 280 nm, which resulted from the release of acid-soluble material as a result of enzymatic hydrolysis of poly(C).

5. Expression and Purification of Recombinant Protein HuS/C Containing a Mutant 18-125 Amino Acid Fragment of Human Ribonuclease A with S(19,20)A and S 123C Amino Acid Substitutions.

The plasmid pETBlue/HuS/C encoding HuS/C recombinant protein designated HuS/C (see, FIG. 2 for schematic representation) was transformed in E. coli Origami B (DE3)pLacI cells. A single selected clone was grown overnight in LB broth (Life Technologies, USA) containing 50 mg/L ampicillin and 34 mg/L chloramphenicol, 12.5 mg/L tetracycline, 15 mg/L kanamycin. The overnight culture was diluted 1:34 into a flask containing 200 ml LB broth with 50 mg/L ampicillin, 34 mg/L chloramphenicol, 12.5 mg/L tetracycline, 15 mg/L kanamycin, and grown shaking at 37° C. until the optical density at 600 nm reached 0.5 measured in a spectrophotometer (Ultraspec 1000, Pharmacia Biotech, USA). At that stage, the expression of HuS/C was induced by the addition of IPTG (Gibco, USA) to a final concentration of 1 mM. The induced culture was grown for 15-16 additional hours and then harvested by centrifugation (25 min., 5000× g). Inclusion body pellet was obtained from a cell culture pellet using BugBuster reagent (Novagen, USA) according to the manufacturer's instructions. The purified inclusion body pellet was washed with high-salt washing buffer containing 20 mM Tris-HCl pH 8.0, 0.5 M NaCl, 5 mM imidazole and than solubilized in 8 M urea by sonication for 30 sec using VirSonic 475 (The Virtis Company, USA) working at 40% output power. The solution was mixed with DTT to a final concentration of 2.5 mM, incubated for 1 hr at 37° C. and than dialyzed for 20-22 hours at 4° C. against buffer containing 0.1 M Tris-Acetate pH 8.6, 0.1 M NaCl, 0.5 M arginine, 1 mM reduced glutathione, 0.4 mM oxidized glutathione. Dialyzed preparation of HuS/C was analyzed by SDS-PAGE and found to be 70-80% pure by Coomassie staining. HuS/C recombinant protein was stored in aliquots at −70° C.

For functional activity testing HuS/C recombinant protein was mixed with varying amounts of human S-peptide Lys-Glu-Ser-Arg-Ala-Lys-Lys-Phe-Gln-Arg-Gln-His-Met-Asp-Ser (SEQ ID NO:2), available from PeptidoGenic Research & Co, USA); and the reconstructed ribonuclease activity was measured as described in Example 1.F.4.

G. Functional Activities of VEGF Fusion Proteins.

1. Cell Lines.

Transformed primary human embryonic kidney cell line (293 cells, ATCC CRL-1573, American Type Culture Collection, USA) were maintained in DMEM (Life Technologies, USA) supplemented with 10% fetal calf serum (Gemini, USA), 2 mM glutamine (Life Technologies, USA), and 1:100 diluted Penicillin-Streptomycin (Life Technologies, USA) at 37° C., 5% CO₂.

Derivatives of 293 cells overexpressing a soluble, secreted extracellular domain of the KDR/flk-1 receptor fused to the Fc portion of human IgG (293/KDR-Fc) obtained from Dr. B. Terman, (A. Einstein School of Medicine, USA) were maintained in DMEM supplemented with 10% fetal calf serum, 2 mM glutamine, 1:100 diluted Penicillin-Streptomycin and 0.2 mg/ml G418 (Life Technologies, USA) at 37° C., 5% CO₂.

293 cells were transfected with pBal/Pst/pur-KDR mammalian cell expression plasmid described in Example 1.A.1, using Mirus Trans IT-LT1 Polyamine Transfection Reagent (PanVera, USA) according to the manufacturer's instructions. Following transfection, cells were cultured for 48 h, split 1:6 into 24-well plates, and after a further 24 h in culture, puromycin (Sigma, USA) was added to a final concentration of 0.375 μg/ml. Several clones were expanded and tested for VEGF165-induced tyrosine autophosphorylation of the KDR/flk-1 receptor as described further. The clone showing the strongest signal in autophosphorylation assay was designated 293/KDR and was used for further experiments. 293/KDR cells were maintained in DMEM supplemented with 10% fetal calf serum, 2 mM glutamine, 1:100 diluted Penicillin-Streptomycin, and 0.375 μg/ml puromycin at 37° C., 5% CO₂.

Porcine endothelial cell line transfected with empty vector designated as PAE, and porcine endothelial cell line overexpressing KDR/flk-1 receptor designated as PAE/KDR were obtained from Dr. B. Terman (A. Einstein School of Medicine, USA). PAE and PAE/KDR cells were maintained in DMEM supplemented with 10% fetal calf serum, 2 mM glutamine, 1:100 diluted Penicillin-Streptomycin at 37° C., 5% CO₂.

2. Binding of VEGF Fusion Proteins to Soluble and Cellular KDR/flk-1 Receptors.

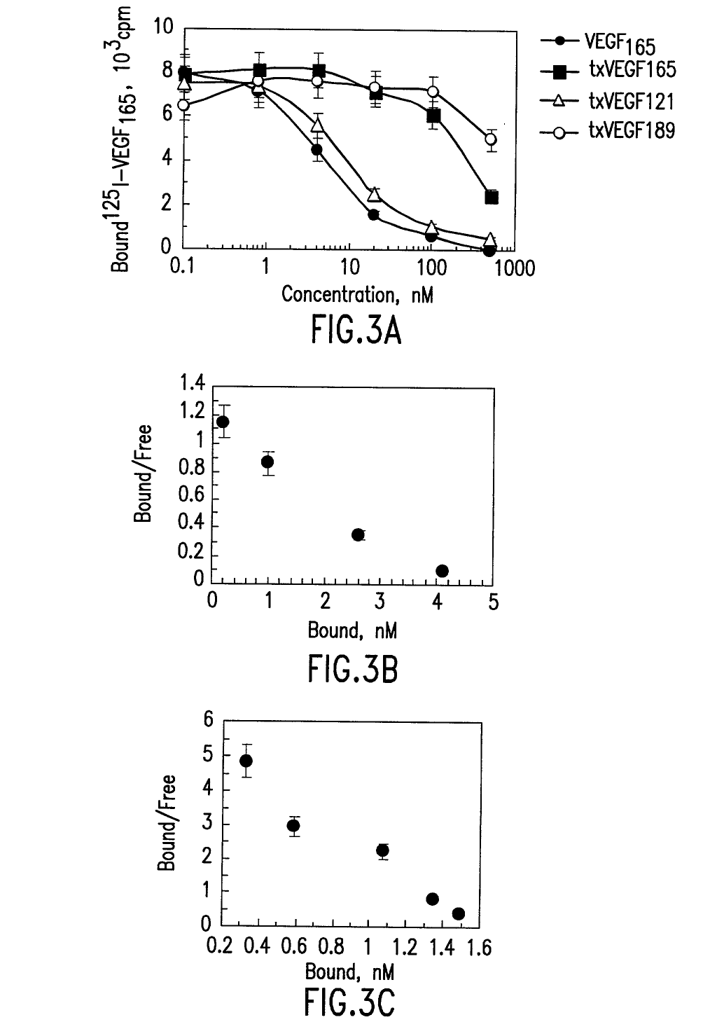

In our approach, fusion targeting proteins should retain functional activity despite presence of large additions to their N- or C-termini. txFVEGF121, txFVEGF165, and txFVEGF189 fusion proteins constructed, expressed, and purified as described above contain an N-terminal extension of 158 amino acid residues. Functional activities of these proteins indicate that VEGF proteins can accommodate large-scale alterations in their N-termini. One test for the functional activity of VEGF fusion proteins is binding to KDR/flk-1 receptors. Soluble KDR-Fc receptors containing the extracellular domain fused to the Fc portion of human IgG were purified from culture fluids of 293/KDR-Fc cells described above. Binding of ¹²⁵I-VEGF165 (Amersham, USA) proteins to soluble KDR-Fc receptors and to cellular KDR/flk-1 receptors on 293/KDR cells was used as a control and was performed as described in the art (Kaplan, et al., 1997). VEGF fusion proteins, txFVEGF121, txFVEGF165, and txFVEGF189 inhibited binding of 1251-VEGF165 to soluble KDR-Fc receptors (FIG. 3A).

Scatchard analysis of ¹²⁵-txFVEGF121 (SibTech, USA) binding to 293/KDR cells established the presence of 2.5 million KDR/flk-1 receptors per cell with Kd of 3.5 nM (FIG. 3B). For comparison, Scatchard analysis of ¹²⁵I-VEGF165 (Amersham, USA) binding to 293/KDR cells established the presence of 2.4 million KDR/flk-1 receptors per cell with Kd of 0.3 nM (FIG. 3C).

3. Induction of the Tyrosine Phosphorylation of Cellular KDR/flk-1 Receptors by VEGF Fusion Proteins.

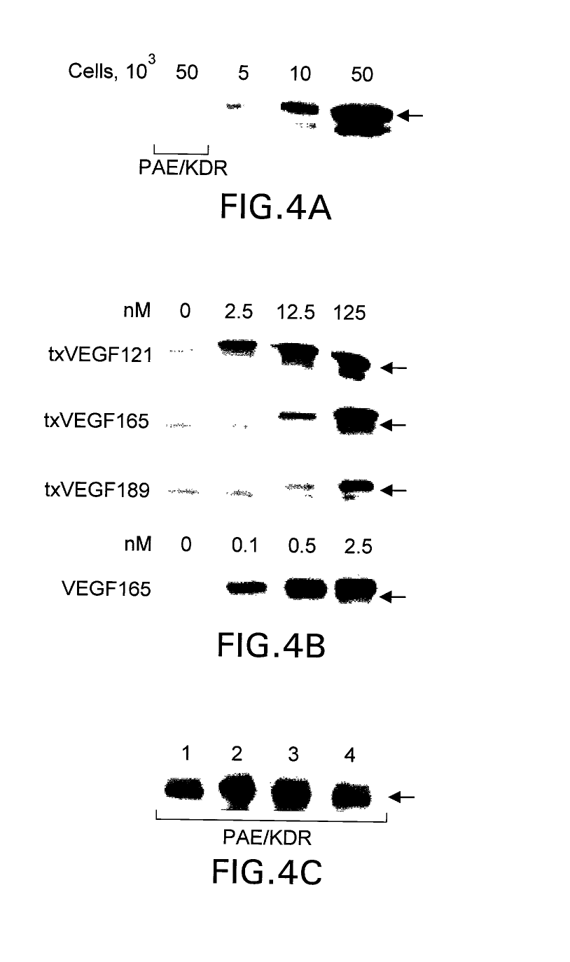

The ability of VEGF fusion proteins to induce tyrosine phosphorylation of cellular KDR/flk-1 receptors, was tested using PAE/KDR and 293/KDR cells. As described above, 293/KDR cells express 2.4 million KDR/flk-1 receptors per cell. According to Western blot analysis of KDR/flk-1 expression in 293/KDR and PAE/KDR cells the latter cells express approximately ten times less KDR/flk-1 receptors than 293/KDR cells (FIG. 4A).

About 50,000 PAE/KDR cells per well or about 120,000 293/KDR cells were plated onto 24-well plates in 1 ml DMEM supplemented with 10% fetal calf serum, 2 mM glutamine, 1:100 diluted Penicillin-Streptomycin and incubated overnight at 37° C., 5% CO₂. Next day, the cells were washed once with phosphate buffered saline and shifted to serum-free DMEM for 4 hours at 37° C. Subsequently, the medium was changed to serum-free DMEM supplemented with 0.1 mM sodium orthovanadate, 100 ng/ml bovine serum albumin, 25 mM HEPES pH 7.2, and the cells were incubated for 20 min at 37° C. followed by a 20-min incubation at 4° C. Then the cells were incubated with VEGF fusion protein for 1 hour at 4° C. followed by 8 min at 37° C. Then the cells were rinsed once with ice-cold phosphate buffered saline containing 0.1 mM sodium orthovanadate, solubilized in sample buffer containing 0.05 M Tris-HCl pH 6.8, 2.5% SDS, 7.5% glycerol, 5 mM EDTA, 50 mM DTT, 0.025% Bromophenol Blue, and analyzed by Western blotting. Cellular proteins were fractionated by SDS-PAGE on 7.5% gels and were transferred to nitrocellulose (BioRad, USA) using a semi-dry system 2117 Multiphor II (LKB, Sweden), as described by the manufacturer. Western blots were processed and probed with anti-phosphotyrosine RC20:HRP conjugate (Transduction Lab, USA) at dilution 1:2,000 according to the manufacturer's instructions. A chemiluminescence-based system (ECL, Amersham, USA) was used for band detection.

High concentrations of VEGF fusion proteins txVEGF121, txVEGF165, and txVEGF189 induced tyrosine phosphorylation of KDR/flk-1 receptors in 293/KDR cells and PAE/KDR cells approximately as efficiently as the correct size VEGF165 (FIGS. 4B, 4C). As described in Example 1.G.2, VEGF fusion proteins have a lower affinity to KDR/flk-1 receptors than the correct size VEGF165. Their efficiency in tyrosine phosphorylation of KDR/flk-1 receptors in 293/KDR and PAE/KDR cells may be due to the high number of the receptors on the cells. Furthermore, it suggests that fusion VEGF proteins may interact more efficiently with cells that overexpress KDR/flk-1 receptors, like endothelial cells at the sites of angiogenesis.

4. Induction of 293/KDR cell Contraction by VEGF Fusion Proteins.

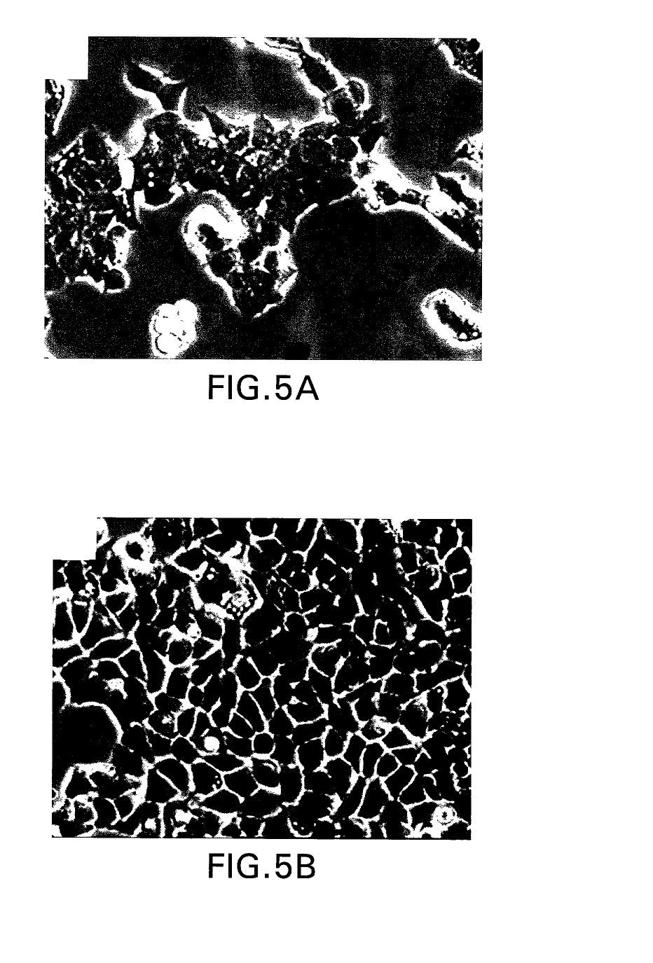

The functional activity of VEGF fusion proteins was further tested in a novel cell contraction assay. 293/KDR cells were grown in the 6-well plates to the stage when they form colonies of flat, tightly packed cells (FIG. 5A) and then were incubated for 3 h at 37° C. with fresh medium, followed by addition of correct size VEGF165 or txFVEGF121, txFVEGF165, or txFVEGF189 fusion protein to the final concentration of 50 nM. After incubation with the proteins for 3 h at 37° C. cells were observed with an Optonics DEI 750 Cooled CCD Camera attached to a Zeiss IM35 microscope. The proteins induced contraction of 293/KDR cells as illustrated for txFVEGF121 proteins (FIG. 5B).

5. Functional Activity of txFVEGF121 Fusion Proteins Associated with S-Protein Fragment of Ribonuclease A.

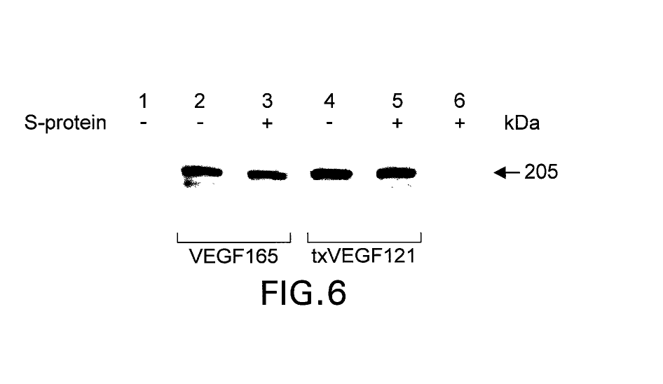

Complexes of txVEGF121 fusion proteins with S-proteins induce tyrosine autophosphorylation of KDR/flk-1 receptors in 293/KDR cells (FIG. 6). For these experiments equimolar amounts of txFVEGF121 and S-protein were mixed to final concentrations of 700 nM in DMEM serum-free medium supplemented with 0.1 mM sodium orthovanadate, 100 ng/ml bovine serum albumin, 25 mM HEPES pH 7.2 medium, and incubated on ice for 15-20 min. The mixture was used in KDR/flk-1 tyrosine autophosphorylation assay performed as described above (Example 1.D.4) using txFVEGF121/S-protein complex at final concentrations of 7 riM. Correct size VEGF165, and equimolar mixture of VEGF165 and S-protein at concentrations of 7 nM were used as controls. Correct size VEGF165 at concentrations of 3.5 nM, and a mixture of VEGF165 at concentrations of 3.5 nM, and S-protein at concentrations of 7 nM were used as controls.

### Example 2

### Preparation of Liposome Carrying S-Protein Fragment of Ribonuclease.

The protocol included conjugation of chemically active NHS-polyethylene glycolated phospholipid to S-protein fragment of ribonuclease and assembly of liposome in the presence of lipid-derivatized S-protein.

1. Conjugation of Polyethylene Glycolated Distearoylphosphatidyletanolamine to S-Protein.

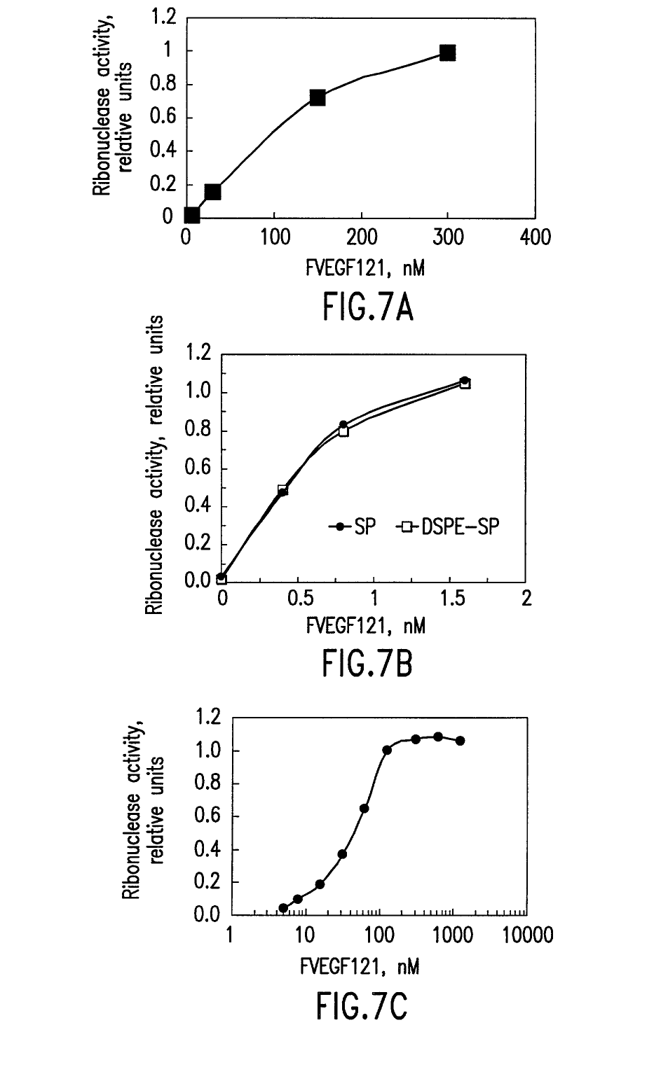

The S-protein fragment of bovine ribonuclease (Sigma, USA) (hereinafter designated as SP) was mixed with NHS -polyethylene glycolated distearoylphosphatidyletanolamine (DSPE-PEG-NHS) (Shearwater Polymers, USA) at the molar ratio 1:0.75 in a conjugation buffer containing 0.1 M NaPi pH 7.2 150 mM NaCl, and incubated at 37° C. for 4 hours under argon. The product, designated DSPE-SP, was purified on a dextran desalting column (Pierce, USA) equilibrated with conjugation buffer. SP and DSPE-SP (4 nM each) were mixed with varying amounts of S-tagged VEGF and the reconstituted ribonuclease activity was measured using S-tag assay (Novagen, USA). DSPE-SP retained ability to bind S-peptides in FVEGF121 fusion proteins (FIG. 7A).

2. Preparation of the Drug-Loaded Liposome Carrying S-Protein.

1,2-Dioleyl-sn-Glycero-3-Phosphocholine and cholesterol (65:35) were lyophilized from cyclohexane and hydrated in HNE buffer containing 10 mM HEPES, 150 mM NaCl, 0.1 mM EDTA, 0.1 mM EGTA, 0.34 mM doxorubicin (Dox), and 13 μM DSPE-SP. Liposomes were prepared using a mini-extruder according to manufacturer's instructions (Avanti, USA). Free Dox and SP were then removed by gel-filtration on Sepharose CL-4B column equilibrated with HNE buffer. The purified doxorubicin-loaded liposomes designated Lip(Dox)-SP, contained 2.5 mM lipids as determined by phosphate assay (Sigma, USA), 14.3 nM Dox as determined by spectrofluorometric assay with excitation at 470 nm and emission at 590 nm, and 5 μM protein as determined by micro BCA protein assay (Pierce, USA). To test ability of Lip(Dox)-SP to interact with S-peptide in the S-tagged fusion proteins, Lip(Dox)-SP was mixed with varying amounts of S-tagged VEGF and the reconstituted ribonuclease activity was measured using S-tag assay kit (Novagen, USA). Lip(Dox)-SP retained ability to bind S-peptides in FVEGF121 fusion proteins (FIG. 7B).

### Example 3

### Preparation of Conjugates of Polyethylenimine With S-Protein Fragment of Ribonuclease.

The conjugation protocol included introduction of SH-groups in polyethylenimine designated as PEI, introduction of maleimide groups in S-proteins designated as SP, and crosslinking of PEI and SP via reactions between SH- and maleimide groups.

1. Modification of PEI.

PEI was modified by 2-iminothiolane (also known as Trout's reagent, available from Pierce Chemical Co., USA) for 20 min at room temperature in a tube under nitrogen in a buffer containing 50 mM triethanolamine, 150 mM NaCl, 1 mM EDTA. Concentrations of PEI and Trout's reagent were 5 mM each. The product, designated PEI-SH, was purified on a dextran desalting column (Pierce, USA) equilibrated with conjugation buffer containing 0.1 M sodium phosphate, pH 7.2 and 150 mM NaCl. The concentration of modified PEI-SH was determined by the Ellman reaction on free SH-groups using Ellman reagent (Pierce, USA) according to the manufacturer's instructions. The content of PEI in each fraction was calculated as a fraction of total Ellman's reactive material multiplied by input PEI.

2. Modification of S-protein.

SP was modified by m-maleimidobenzoyl-N-hydroxysulfosuccinimide ester, also known as sulfo-MBS for 30 min at room temperature in a conjugation buffer containing 0.1 M sodium phosphate, pH 7.2, and 150 mM NaCl. The ratio of sulfo-MBS to SP was varied. The product was purified on a dextran desalting column (Pierce, USA) equilibrated with conjugation buffer containing 0.1 M sodium phosphate, pH 7.2, 150 mM NaCl. Eluted fractions were tested for the presence of material with absorbance at 280 nm. The content of SP in each fraction was calculated as a fraction of total 280 nm absorbing material.

Table 1 illustrates ribonuclease activity of complexes of S-peptide with S-protein modified with various amounts of m-maleimidobenzoyl-N-hydroxysulfosuccinimide ester (sulfoMBS). Ribonuclease activity was measured with S-tag Rapid Assay Kit (Novagen, USA). Modification of SP with increasing amounts of sulpho-MBS gradually inhibited ability of modified SP to reconstruct ribonuclease activity. For experiments described in other examples modified SP fragment was prepared by incubating SP with a two-fold excess of sulfo-MBS at final concentrations of 1.32 mM sulfo-MBS and 0.66 mM SP.

TABLE 1
Ribonuclease Activity of MBS-modified S-Protein

| SulphoMBS, Molar Excess | Ribonuclease Activity (%) |
|---|---|
| none | 100 |
| 2 | 57 |
| 10 | 38 |
| 20 | 36 |
| 40 | 25 |

3. Crosslinking of PEI-SH and Modified S-Protein.

Modified S-protein at a concentration of 127 μM, obtained as described above, was incubated with equimolar amounts of modified PEI-SH obtained as described above for 4 hours at room temperature in a conjugation buffer. The conjugate designated as PEI-SP was separated from unreacted modified S-protein on a G-100 Sepharose column equilibrated with phosphate buffered saline. Fractions containing peak of PEI-SP were combined, aliquoted, and stored at −20° C. PEI-SP retained the ability to bind to S-peptides, and to S-peptides in FVEGF121 fusion proteins (FIG. 7A).

4. Crosslinking of Other Therapeutic, Diagnostic and Research Compounds to S-Protein.

Other therapeutic, diagnostic and research compounds and carriers of therapeutic, diagnostic and research compounds can be readily crosslinked to S-protein. Such carriers include but are not limited to natural or synthetic polymers and co-polymers, such as dextran and other polysaccharides, polylysine, polyethylenimine, poly(vinyl alcohol), poly(divinyl) ether-co-maleic anhydrdride, poly(ethylene glycol), poly(methyl methacrylates), polyanhydrides, polyesters, polyacrylic acids, polyurethanes,N-(2-hydroxypropyl)methacrylamide, derivatized polymers and co-polymers, liposomes, various viral and bacteriophage particles, and various manufactured beads and nanoparticles. For example, various phospholipids, various phospholipids conjugated to polyethyleneglycol, phospholipids conjugated to chelating compounds, boron compounds, chelating compounds, and the like, are now commercially available in forms that contain chemically active groups useful for chemical conjugation either directly to S-proteins, or to bifunctional crosslinking agent introduced into S-proteins either randomly or through specific amino acid residues, such as an additional cysteine residue in Cys-mutants of S-protein described in Example 1.

### Example 4

### Functional Activity of Complexes of Lip(DOX)-SP with FVEGF121.

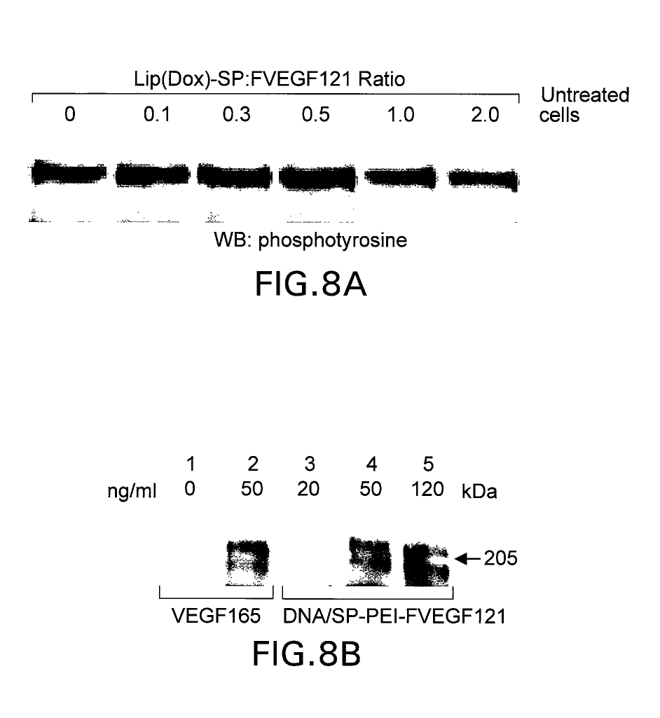

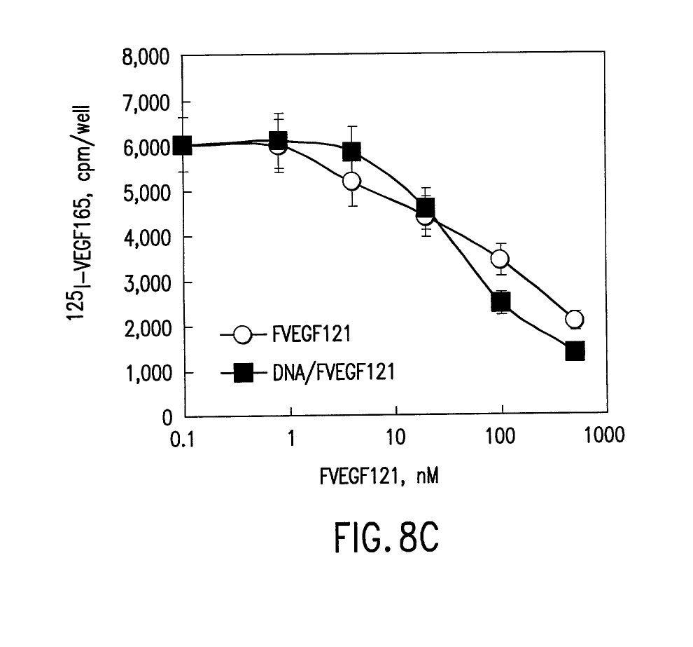

Lip(DOX)-SP-FVEGF121 complexes were tested for the ability to induce tyrosine phosphorylation of KDR/flk-1 receptors in 293/KDR cells. VEGF was mixed with increasing amounts of Lip(Dox)-SP-FVEGF121, incubated on ice for 15 min, and added to cells, preincubated in serum-free medium at 4° C., to a final VEGF concentration of 12.5 nM. After addition of Lip(Dox)-SP-FVEGF121 cells were incubated for 1 hr at 4° C., then for 10 min at 37° C., lysed and analyzed by Western blot analysis as described in Example 1.G.3 Lip(DOX)-SP-FVEGF121 complexes induced tyrosine phosphorylation of KDR/flk-1 receptors (FIG. 8A).

### Example 5

### Preparation of Complexes of DNA with PEI-SP Conjugates and FVEGF121.

1. Assembly of Complexes.

PEI-SP conjugates were mixed with FVEGF121 in serum-free DMEM at the desired ratio and incubated on ice for 20 min. The mixture was added to an ice-cold solution of pRL-tk plasmid in serum-free DMEM at the desired ratio and incubated for additional 20 min on ice. Complexes were designated DNA/PEI-SP-FVEGF121 and were prepared fresh for each experiment. In separate experiments DNA/PEI-SP-FVEGF121 complexes were prepared at room temperature and found to be less efficient in DNA delivery than DNA/PEI-SP-FVEGF121 complexes prepared on ice. In additional experiments, when PEI-SP conjugates were first mixed with DNA and then VEGF was added to the reaction mixture, the resulting DNA/PEI-SP-FVEGF121 complexes were inefficient in DNA delivery.

2. Functional Activity of DNA/PEI-SP-FVEGF121 Complexes.

DNA/PEI-SP-FVEGF121 complexes obtained as described above were tested for the ability to induce tyrosine phosphorylation of KDR/flk-1 receptors in 293/KDR cells. DNA/PEI-SP-FVEGF121 complexes induced tyrosine phosphorylation of KDR/flk-1 receptors in a dose-dependent manner with an efficiency comparable to that of correct size VEGF165 (FIG. 8B).

DNA/PEI-SP-FVEGF121 complexes were also tested for the ability to compete with ¹²⁵I VEGF165 (Amersham, USA) for binding to KDR/flk-1 receptors in 293/KDR cells. 293/KDR cells were seeded in 24-well plates and 24 hours later the medium was changed to serum-free DMEM supplemented with 25 mM HEPES pH 7.5. ¹²⁵I-VEGF165 (40,000 cpm/well) was mixed with FVEGF121 or DNA/PEI-SP-FVEGF121 and added to cells at the indicated concentrations. After 90 min at room temperature medium was removed and cells were washed twice with cold phosphate buffer saline containing 1% bovine serum albumin and once with ice-cold phosphate buffer saline containing 1% bovine serum albumin and 0.4 M NaCl. Cells were solubilized with 0.25 ml of 1% Triton X-100, 0.25 M NaCl, 10 mM Tris-HCl for 30 min at room temperature and radioactivity was counted in a gamma-counter. This experiment established that DNA/PEI-SP-FVEGF121 complexes competed with ¹²⁵I-VEGF165 as efficiently as FVEGF121 fusion proteins (FIG. 8C).

### Example 6

### Liposome Delivery by DNA/PEI-SP-FVEGF121 Complexes

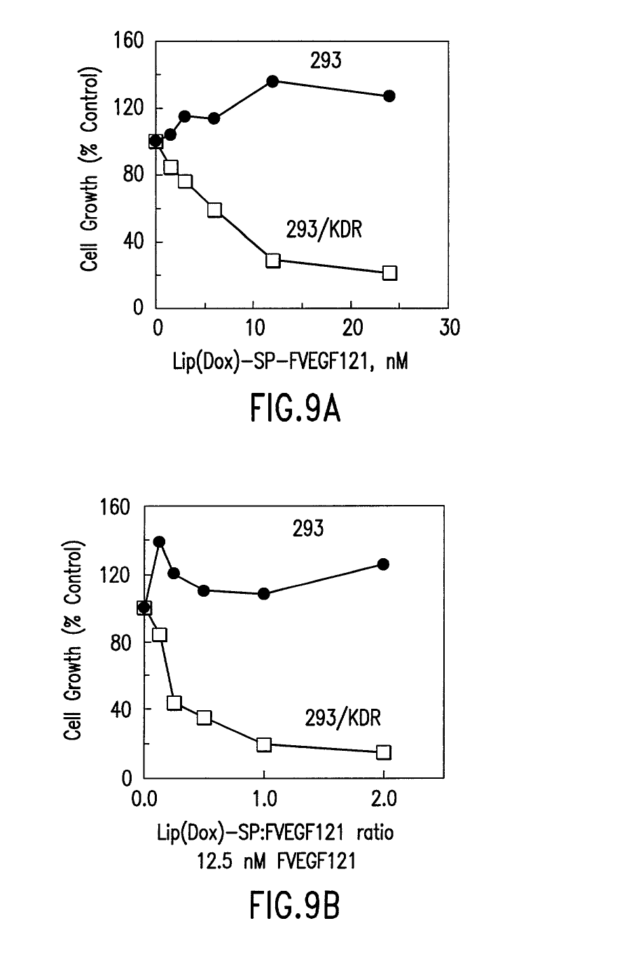

293 and 293/KDR cells were plated in 24-well plates at 105 cells/well and maintained at 37° C. and 5% CO₂ in medium described in Example 1.G.1. Lip(Dox)-SP-FVEGF121 complexes with 1:1 (FIG. 9A) or varying (FIG. 9B) Lip(Dox)-SP:FVEGF 121 molar ratio were added to cells in fresh culture medium 20 hrs later. Control cells were treated with equal amounts of Lip(Dox)-SP without FVEGF121. After 16-18 hours the medium was replaced with fresh culture medium. Cells were counted in a Coulter Counter after 4-days incubations.

Lip(Dox)-SP-FVEGF121 complexes inhibited growth of 293/KDR cells that overexpress KDR/flk-1 receptors with IC₅₀˜5 nM, while growth of 293 cells that do not have the receptors was not inhibited (FIG. 9A). The inhibitory activity of Lip(Dox)-SP-FVEGF121 complexes required the presence of FVEGF121 in this complexes and a single molecule of FVEGF121 per complex was sufficient to achieve almost 100% of inhibitory effect (FIG. 9B).

### Example 7

### DNA Delivery by DNA/PEI-SP-FVEGF121 Complexes

A. Methods

1. DNA Delivery to Cells.

293 and 293/KDR cells were plated in 24-well plates at 10⁵ cells/well and maintained for 16-20 hours at 37° C. and 5% CO₂ in medium described in Example 1.G.1. Two protocols for DNA delivery were employed. In the first protocol DNA/PEI-SP-FVEGF121 complexes were added directly to cells maintained in complete medium for 16-20 hours. In the second protocol, after being kept for 16-20 hours at 37° C. and 5% CO₂ in complete medium cells were incubated for 4 hours at 37° C. and 5% CO₂ in serum-free DMEM supplemented with 25 mM HEPES, pH 7.2. Plates were then shifted to 4° C. and incubated for 20 min, followed by addition of ice-cold solutions of DNA/PEI-SP-FVEGF121 complexes and incubated for an additional 40 min at 4° C. Plates were then we shifted to 37° C. and 5% CO₂ and after a 4-hour incubation medium was supplemented with fetal bovine serum to a final concentration of 10%.

As a control for transfectability of 293 and 293/KDR cells, in each experiment cells were transfected with a mixture of DNA and PEI at a N/P ratio of 8, where N is the concentration of PEI amino groups and P is the concentration of DNA phosphate groups.

2. Measurements of DNA Delivery to Cells.

The reporter plasmid pRL-tk for expression of luciferase in mammalian cells (Promega, USA) was used for delivery to cells. Luciferase activity was measured 24 hours after delivery. Luciferase activity in cell lysates was quantitated using a commercially available Dual-Luciferase Reporter Assay System (Promega, USA) according to the manufacturer's instructions.

In separate experiments with triplicate wells we found that variations in the levels of luciferase expression within the same experiment were less than 20%. We therefore used single wells for the majority of experimental points.

All experiments were repeated 2-3 times. According to our data and in agreement with reports from other groups (Bousiffet al., 1996, Behr, 1995; Zanta et al., 1997; Kircheis et al., 1997) luciferase expression varied several fold between experiments reflecting, most likely, some subtle changes in the properties of DNA complexes (Roland, 1998). Representative results are presented herein.

B. DNA Delivery by DNA/PEI-SP-FVEGF121 Complexes

1. Preferential DNA Delivery by DNA/PEI-SP-FVEGF121 Complexes Into 293/KDR Cells.

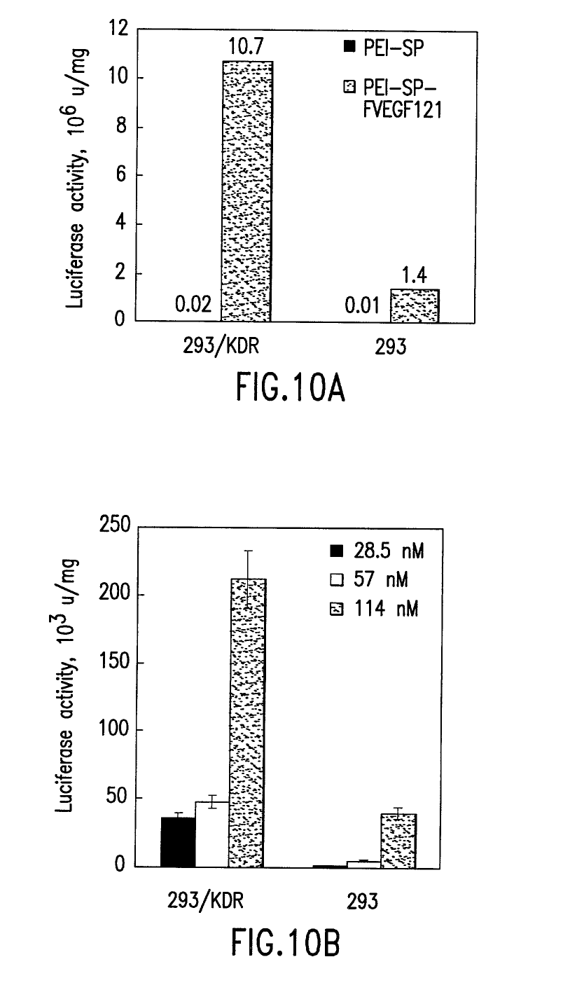

Using the protocols described in Example 4.A.1, DNA/PEI-SP-FVEGF121 complexes were employed to deliver DNA to 293/KDR and 293 cells. As a control, the same amounts of DNA and PEI-SP were mixed and added to cells. Intracellular DNA delivery measured by luciferase expression was four to five times higher for 293/KDR cells that overexpress KDR/flk-1 receptors than to 293 cells that do not have the receptors (FIGS. 10A and 10B). This difference was observed when binding of DNA/PEI-SP-FVEGF121 complexes were initiated in serum-free medium at 4° C. and when DNA/PEI-SP-FVEGF121 complexes were added directly to complete medium at 37° C. Delivery of DNA by PEI-SP complexes alone was negligible at the chosen N/P ratio (FIG. 10B).

2. Contribution of KDR/flk-1 Receptor-Mediated Pathway to DNA Delivery by DNA/PEI-SP-FVEGF121 Complexes Into 293/KDR Cells.

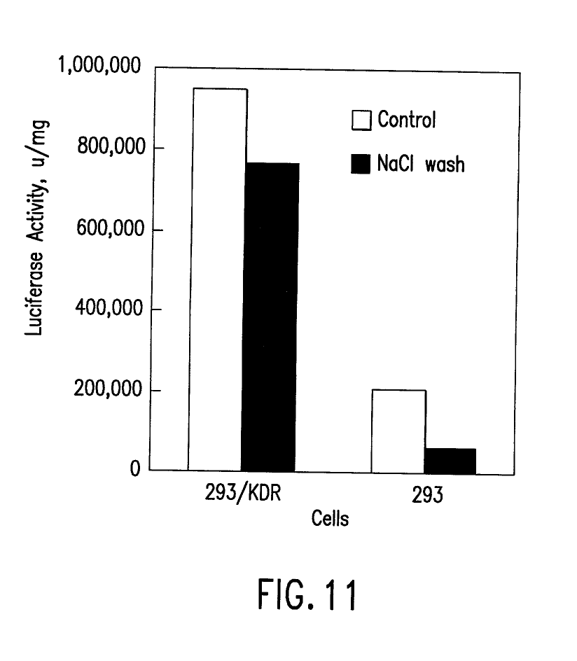

Since we employed FVEGF121 lacking a heparin-binding domain we did not expect the FVEGF121 moiety in DNA/SP-PEI-FVEGF121 to bind to cell surface heparin sulfate proteoglycans. However, it was shown previously that poly-L-Lysine or cationic lipid-DNA complexes may enter cells after binding to negatively charged cell surface proteoglycans (Mislick & Baldeschwieler 1996). It was reasonable to assume that DNA/SP-PEI-FVEGF121 complexes may also bind to cell surface proteoglycans via the PEI moiety. To test this hypothesis DNA/SP-PEI-FVEGF121 were bound to 293 and 293/KDR cells at 4° C. and then cells were washed with phosphate buffered saline containing 0.4 M NaCl prior to incubation at 37° C. The salt wash decreased expression of luciferase in 293/KDR cells only by ˜20%, while expression in 293 cells diminished by 70% (FIG. 11). These results indicate that the majority of DNA enters 293/KDR cells via KDR/flk-1 receptor-mediated pathways.

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patent applications, patents, and other publications cited herein are incorporated by reference in their entireties.

What is claimed is:

1. A molecular delivery vehicle for delivery of therapeutic, diagnostic, or research compounds to a target, comprising:
(a) a carrier for carrying said compounds;
(b) an adapter covalently linked to said carrier; and
(c) a targeting protein comprising a recognition portion and a targeting portion, said recognition portion capable of binding to said adapter, said targeting portion capable of binding to said target.

2. The molecular delivery vehicle of claim 1, wherein said target is attached to a natural or artificial surface.

3. The molecular delivery vehicle of claim 1, wherein said target is a cell surface receptor or a cell surface antigen.

4. The molecular delivery vehicle of claim 1, wherein said adapter functions as said target.

5. The molecular delivery vehicle of claim 1, wherein said carrier comprises a polymer.

6. The molecular delivery vehicle of claim 1, wherein said carrier is selected from the group consisting of polysaccharides, polylysine, polyethylenimine, poly(vinyl alcohol), poly(divinyl) ether-co-maleic anhydrdride, poly(ethylene glycol), poly(methyl methacrylates), polyanhydrides, polyesters, polyacrylic acids, polyurethanes,N-(2-hydroxypropyl)methacrylamide, derivatized polyethylenglycoles, co-polymers and derivatized co-polymers, liposomes and derivatized liposomes, dendrimers and derivatized dendrimers, viral and bacteriophage particles, bids, nanoparticles, and combinations thereof.

7. The molecular delivery vehicle of claim 1, wherein said therapeutic, diagnostic, or research compounds are selected from the group consisting of nucleic acids, peptides, proteins, viruses, viral particles employed for gene delivery, chemotherapeutic agents, paramagnetic agents, radioactive agents, fluorogenic agents, and combinations thereof.

8. The molecular delivery vehicle of claim 1, wherein said adapter is selected from the group consisting of a wild type or mutant S-protein fragment of bovine or human ribonuclease A, cellulose, calmodulin, and streptavidin.

9. The molecular delivery vehicle of claim 1, wherein said targeting portion of said targeting protein is selected from the group consisting of cytokines, growth factors, peptide hormones, antibodies, fusion proteins, and combinations thereof.

10. The molecular delivery vehicle of claim 1, wherein said targeting portion of said targeting protein is vascular endothelial growth factor 121.

11. The molecular delivery vehicle of claim 1, wherein said recognition portion of said targeting protein is an S-peptide fragment of bovine or human ribonuclease A.

12. The molecular delivery vehicle of claim 1, wherein said recognition portion of said targeting protein is located at the N-terminus of s aid targeting protein.

13. The molecular delivery vehicle of claim 1, wherein said target is a receptor found on a cell selected from the group consisting of cells expressing receptors for vascular endothelial growth factor.

14. The molecular delivery vehicle of claim 1, wherein said targeting protein further comprises a spacer peptide positioned between said recognition portion and said targeting portion.

15. A pharmaceutical composition, comprising:
(1) a pharmaceutically acceptable carrier; and
(2) a pharmaceutically effective amount of a molecular delivery vehicle for delivery of therapeutic, diagnostic, or research compounds to a target, comprising:
(a) a carrier for carrying said compounds;
(b) an adapter covalently linked to said carrier; and
(c) a targeting protein comprising a recognition portion and a targeting portion, said recognition portion capable of binding to said adapter, said targeting portion capable of binding to said target.

16. The pharmaceutical composition of claim 15, wherein said target is attached to a natural or artificial surface.

17. The pharmaceutical composition of claim 15, wherein said target is a cell surface receptor or a cell surface antigen.

18. The pharmaceutical composition of claim 15, wherein said adapter functions as said target.

19. The pharmaceutical composition of claim 15, wherein said carrier comprises a polymer.

20. The pharmaceutical composition of claim 15, wherein said carrier is selected from the group consisting of polysaccharides, polylysine, polyethylenimine, poly(vinyl alcohol), poly(divinyl) ether-co-maleic anhydrdride, poly(ethylene glycol), poly(methyl methacrylates), polyanhydrides, polyesters, polyacrylic acids, polyurethanes,N-(2-hydroxypropyl)methacrylamide, derivatized polyethylenglycols, co-polymers and derivatized co-polymers, liposomes and derivatized liposomes, dendrimers and derivatized dendrimers, viral and bacteriophage particles, bids, nanoparticles, and combinations thereof.

21. The pharmaceutical composition of claim 15, wherein said therapeutic, diagnostic, or research compounds are selected from the group consisting of nucleic acids, peptides, proteins, viruses, viral particles employed for gene delivery, chemotherapeutic agents, paramagnetic agents, radioactive agents, fluorogenic agents, and combinations thereof.

22. The pharmaceutical composition of claim 15, wherein said adapter is selected from the group consisting of a wild-type or mutant S-protein fragment of bovine or human ribonuclease A, cellulose, calmodulin, and streptavidin.

23. The pharmaceutical composition of claim 15, wherein said targeting portion of said targeting protein is selected from the group consisting of cytokines, growth factors, peptide hormones, antibodies, fusion proteins, and combinations thereof.

24. The pharmaceutical composition of claim 18, wherein said targeting portion of said targeting protein is vascular endothelial growth factor 121.

25. The pharmaceutical composition of claim 15, wherein said recognition portion of said targeting protein is an S-peptide fragment of bovine or human ribonuclease A.

26. The pharmaceutical composition of claim 15, wherein said recognition portion of said targeting protein is located at the N-terminus of said targeting protein.

27. The pharmaceutical composition of claim 15, wherein said target is a receptor found on a cell selected from the group consisting of cells expressing receptors for vascular endothelial growth factor.

28. The pharmaceutical composition of claim 15, wherein said targeting protein further comprises a spacer peptide positioned between said recognition portion and said targeting portion.

29. The pharmaceutical composition of claim 15, wherein said pharmaceutically acceptable carrier is selected from the group consisting of water, gelatin, lactose, starch, magnesium stearate, talc, plant oils, gums, alcohol, petroleum jelly, buffered saline, and combinations thereof.

30. An article of manufacture comprising packaging material and a pharmaceutical agent contained within said packaging material, wherein said pharmaceutical agent is therapeutically effective for treating pathophysiological conditions that depend on cells that can be detected or affected via target-mediated delivery of therapeutic or diagnostic compounds and wherein said packaging material comprises a label which indicates that the pharmaceutical agent can be used for treating pathophysiological conditions that depend on cells that can be detected or affected via target-mediated delivery of therapeutic or diagnostic compounds, and wherein said pharmaceutical agent comprises a pharmaceutically effective amount of a molecular delivery vehicle for delivery of therapeutic, diagnostic, or research compounds to a target, comprising:
(a) a carrier for carrying said compounds;
(b) an adapter covalently linked to said carrier; and
(c) a targeting protein comprising a recognition portion and a targeting portion, said recognition portion capable of binding to said adapter, said targeting portion capable of binding to said target;
in a pharmaceutically acceptable carrier.

31. A method for delivering therapeutic, diagnostic, or research compounds to a target, comprising the step of: administering a pharmaceutical composition comprising:
(1) a pharmaceutically acceptable carrier; and
(2) a pharmaceutically effective amount of a molecular delivery vehicle for delivery of compounds to a target, comprising:
(a) a carrier for carrying said compounds;
(b) an adapter covalently linked to said carrier; and
(c) a targeting protein comprising a recognition portion and a targeting portion, said recognition portion capable of binding to said adapter, said targeting portion capable of binding to said target.

32. The method of claim 31, wherein said target is attached to a natural or artificial surface.

33. The method of claim 31, wherein said target is a target is a cell surface receptor or a cell surface antigen.

34. The method of claim 31, wherein said adapter functions as said target.

35. The method of claim 31, wherein said carrier comprises a polymer.

36. The method of claim 31, wherein said carrier is selected from the group consisting of polysaccharides, polylysine, polyethylenimine, poly(vinyl alcohol), poly(divinyl) ether-co-maleic anhydrdride, poly(ethylene glycol), poly(methyl methacrylates), polyanhydrides, polyesters, polyacrylic acids, polyurethanes,N-(2-hydroxypropyl)methacrylamide, derivatized polyethylenglycols, co-polymers, derivatized co-polymers, liposomes and derivatized liposomes, dendrimers and derivatized dendrimers, viral and bacteriophage particles, bids, nanoparticles, and combinations thereof.

37. The method of claim 31, wherein said therapeutic, diagnostic, or research compounds are selected from the group consisting of nucleic acids, peptides, proteins, viruses, viral particles employed for gene delivery, chemotherapeutic agents, paramagnetic agents, radioactive agents, fluorogenic agents, and combinations thereof.

38. The method of claim 31, wherein said adapter is selected from the group consisting of wild-type or mutant S-protein fragment of bovine or human ribonuclease A, cellulose, calmodulin, and streptavidin.

39. The method of claim 31, wherein said targeting portion of said targeting protein is selected from the group consisting of cytokines, growth factors, peptide hormones, antibodies, fusion proteins, and combinations thereof.

40. The method of claim 31, wherein said targeting portion of said targeting protein is vascular endothelial growth factor 121.

41. The method of claim 31, wherein said recognition portion of said targeting protein is an S-peptide fragment of bovine or human ribonuclease A.

42. The method of claim 31, wherein said recognition portion of said targeting protein is located at the N-terminus of said targeting protein.

43. The method of claim 31, wherein said target is a receptor found on a cell selected from the group consisting cells expressing receptors for vascular endothelial growth factor.

44. The method of claim 31, wherein said targeting protein further comprises a spacer peptide positioned between said recognition portion and said targeting portion.

45. The method of claim 31, wherein said pharmaceutically acceptable carrier is selected from the group consisting of water, gelatin, lactose, starch, magnesium stearate, talc, plant oils, gums, alcohol, petroleum jelly, buffered saline, and combinations thereof.

46. An isolated nucleic acid sequence, comprising (i) a first nucleic acid sequence segment encoding an S-peptide, and (ii) a second nucleic acid sequence segment encoding a full-length or mutated isoform of human vascular endothelial growth factor (VEGF), wherein said isolated nucleic acid sequence codes for a fusion protein which specifically binds to receptors for vascular endothelial growth factor recognized by the polypeptide encoded by said second nucleic acid sequence segment.

47. An isolated polypeptide encoded by the isolated nucleic acid sequence of claim 46.

48. An expression vector, comprising control elements operably linked to the nucleic acid sequence of claim 46.

49. A transformed bacterial cell containing the expression vector of claim 48.

50. Isolated nucleic acid sequences encoding wild-type and mutant human and bovine S-proteins.

51. The polypeptides encoded by the isolated nucleic acid sequences of claim 50.

52. Expression vectors, comprising control elements operably linked to the nucleic acid sequences of claim 50.

53. Transformed bacterial cells containing the expression vectors of claim 52.

54. An isolated nucleic acid sequence, comprising (i) a first nucleic acid sequence segment encoding S-protein, and (ii) a second nucleic acid sequence encoding a full-length or mutated isoform of human vascular endothelial growth factor (VEGF), wherein said isolated nucleic acid sequence codes for a fusion protein which specifically binds to receptors for vascular endothelial growth factor recognized by the polypeptide encoded by said second nucleic acid sequence segment.

55. An isolated polypeptide encoded by the isolated nucleic acid sequence of claim 54.

56. An expression vector, comprising control elements operably linked to the nucleic acid sequence of claim 54.

57. A transformed bacterial cell containing the expression vector of claim 56.


SEQUENCE LISTING (30 sequences)

SEQ ID NO: 1 - protein, 15 aa, Human
KETAAAKFERQHMDS

SEQ ID NO: 2 - protein, 15 aa, Human
KESRAKKFQRQHMDS

SEQ ID NO: 3 - DNA, 26 bp, Artificial Sequence
  Synthetic nucleotide probe.
taaggcctatggcagaaggaggaggg

SEQ ID NO: 4 - DNA, 27 bp, Artificial Sequence
  Synthetic nucleotide probe.
actcgagtcaccgcctcggcttgtcac

SEQ ID NO: 5 - DNA, 27 bp, Bovine
atgagcagctccaactactgtaaccag

SEQ ID NO: 6 - DNA, 24 bp, Bovine
atgacttccgctgccagcagctcc

SEQ ID NO: 7 - DNA, 24 bp, Bovine
atgtccgctgccagcagctccaac

SEQ ID NO: 8 - DNA, 26 bp, Bovine
atggctgccagcagctccaactactg

SEQ ID NO: 9 - DNA, 28 bp, Bovine
atggccagcagctccaactactgtaacc

SEQ ID NO: 10 - DNA, 27 bp, Bovine
atgagcagctccaactactgtaaccag

SEQ ID NO: 11 - DNA, 28 bp, Bovine
ctacactgaagcatcaaagtggactggc

SEQ ID NO: 12 - DNA, 28 bp, Bovine
ctacactgaacaatcaaagtggactggc

SEQ ID NO: 13 - DNA, 34 bp, Bovine
ctacactgagcaagcatcaaagtggactggcacg

SEQ ID NO: 14 - DNA, 26 bp, Human
atggactcgagcccgtcttcttcttc

SEQ ID NO: 15 - DNA, 26 bp, Human
atggctgccagcagctccaactactg

SEQ ID NO: 16 - DNA, 27 bp, Human
atgtcttcttctacgtactgcaaccag

SEQ ID NO: 17 - DNA, 25 bp, Human
tcaagagtcttcaacagacgcgtcg

SEQ ID NO: 18 - DNA, 25 bp, Human
tcaagagtcttcacaagacgcgtcg

SEQ ID NO: 19 - DNA, 33 bp, Human
tcaagagtcgcattcaacagacgcgtcgaaatg

SEQ ID NO: 20 - DNA, 36 bp, Human
atgagcgctgcctcttcttctacgtactgcaaccag

SEQ ID NO: 21 - DNA, 39 bp, Human
atgtccgctgccagcagctctacgtactgcaaccagatg

SEQ ID NO: 22 - DNA, 47 bp, Human
atgtccgctgccagcagctccaactactgcaaccagatgatgcgtcg

SEQ ID NO: 23 - DNA, 30 bp, Human
tcattcaacagacgcgtcgaaatgaaccgg

SEQ ID NO: 24 - DNA, 30 bp, Human
ctattcaacacacgcgtcgaaatgaaccgg

SEQ ID NO: 25 - DNA, 33 bp, Bovine
aacatgcatatgagcacttccgctgccagcagc

SEQ ID NO: 26 - DNA, 34 bp, Bovine
tacggtacccactgaagcatcaaagtggactggc

SEQ ID NO: 27 - DNA, 53 bp, Human
  CDS (1)...(53)
atgaaagaatctagacgtaaaaaatttcaacgtcaacacatggactctggtac

SEQ ID NO: 28 - protein, 17 aa, Human
MKESRRKKFQRQHMDSG

SEQ ID NO: 29 - DNA, 48 bp, Human
cagagtccatgtgttgacgttgaaattttttacgtctagattctttca

SEQ ID NO: 30 - protein, 7 aa, Bovine
GTDDDDK